(19) United States
(12) Patent Application Publication
(10) Pub. No.: US 2015/0044155 A1
(43) Pub. Date: Feb. 12, 2015

(54) HAIR PREPARATIONS COMPRISING HYDROXY-TERMINATED ORGANOPOLYSILOXANE(S) AND CONDITIONER(S)

(71) Applicant: Henkel AG & Co. KGaA, Duesseldorf (DE)

(72) Inventors: Erik Schulze zur Wiesche, Hamburg (DE); Markus Semrau, Langenfeld (DE)

(21) Appl. No.: 14/521,638

(22) Filed: Oct. 23, 2014

Related U.S. Application Data
(63) Continuation of application No. PCT/EP2013/053822, filed on Feb. 26, 2013.

(30) Foreign Application Priority Data
Apr. 26, 2012 (DE) 10 2012 206 949.6

(51) Int. Cl. A61K 8/892; A61Q 5/12

(52) U.S. Cl. CPC A61K 8/892; A61Q 5/12; A61K 2800/59
USPC 424/70.12

(57) ABSTRACT

Cosmetic compositions which contain at least one conditioning agent selected from synthetic oils, mineral oils, plant oils, fluorinated or perfluorinated oils, natural or synthetic waxes, compounds of the ceramide type, carboxylic acid esters, silicones differing from the silicones of formulae (I), anionic polymers, nonionic polymers, cationic polymers, amphoteric polymers, cationic proteins, cationic protein hydrolysates, cationic interfacially active substances and mixtures thereof and at least one hydroxy-terminated organopolysiloxane of formula (I). The cosmetic compositions improve numerous properties of the body surfaces, in particular hair, treated therewith and, in addition to improved combability and improved handle, in particular lead to a reduction in the contact angle of the hair.

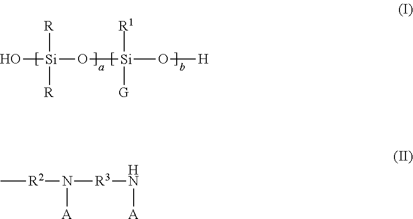

### CROSS-REFERENCE TO RELATED APPLICATIONS

The present application is a continuation of International Patent Application No. PCT/EP2013/053822, filed Feb. 26, 2013 and entitled “HAIR PREPARATIONS COMPRISING HYDROXY-TERMINATED ORGANOPLOYSILOXANE(S) AND CONDITIONER(S)”, which claims priority to DE 10 2012 206 949.6, filed Apr. 26, 2012, by which both are hereby incorporated by reference in their entirety.

### FIELD OF THE INVENTION

The present invention generally relates to hair treatment agents which contain specific substituted silicone(s) and to the use of these agents for cleaning and/or conditioning hair.

### BACKGROUND OF THE INVENTION

Conditioning agents for keratinic fibers have an influence on the natural structure and properties of the hair. Accordingly, after such treatments, for example the hair's wet and dry combability and its hold and body may be optimized or the hair may be protected from increased splitting. It has therefore long been usual to subject the hair to a special post-treatment. In this case, hair is conventionally treated with special active ingredients, for example quaternary ammonium salts, or specific polymers in the form of a rinse. Depending on formulation, this treatment improves the hair's combability, hold and body and reduces splitting.

In recent times, “combined preparations” have been developed in order to reduce the complexity of conventional multistage methods, in particular for direct use by consumers. In addition to the conventional components, for example for cleaning hair, these preparations also contain active substances which were previously found only in hair post-treatment agents. The consumer is thus saved one stage during use; at the same time packaging costs are reduced because one product less is used.

Known active substances are, however, not capable of adequately meeting all requirements. A need accordingly still remains for active substances or active substance combinations for cosmetic agents which have good hair care properties and good biological degradability. In particular in surfactant- and/or electrolyte-containing formulations, there is a need for additional conditioning active ingredients which can be straightforwardly incorporated into known formulations and do not have their action attenuated therein due to incompatibility with other ingredients.

Silicones and, among these, amino-functional silicones are known as conditioning substances in hair treatment agents, and corresponding products are widespread on the market. There is, however, still a requirement to improve the effects achieved, in particular with regard to handle, combability, softness and volume of the hair or hairstyle and to reduce input quantities.

The object of the present invention was to provide silicone-containing hair treatment agents which impart better properties to hair treated therewith than do hair treatment agents with known amodimethicones. Furthermore, it should be possible to achieve equally good or better effects even with distinctly reduced input quantities. In particular, the products should improve the handle, combability, softness and volume of the hair or hairstyle and distinctly minimize the contact angle of water drops which come into contact with the treated hair, said contact angle being a measure of product performance.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic composition containing in a cosmetically acceptable medium: (a) at least one conditioning agent selected from synthetic oils, mineral oils, plant oils, fluorinated or perfluorinated oils, natural or synthetic waxes, compounds of the ceramide type, carboxylic acid esters, silicones differing from the silicones of formulae (I), anionic polymers, nonionic polymers, cationic polymers, amphoteric polymers, cationic proteins, cationic protein hydrolysates, cationic interfacially active substances and mixtures of these various compounds; and (b) at least one hydroxy-terminated organopolysiloxane of general formula (I),

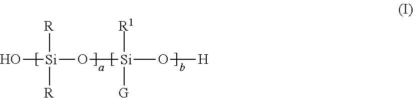

Wherein R means a monovalent unsubstituted or halo-substituted hydrocarbon residue with 1 to 20 carbon atoms; R1 means a monovalent unsubstituted or halo-substituted hydrocarbon residue with 1 to 20 carbon atoms, —OR4 or —OH; R4 means an alkyl residue with 1 to 6 carbon atoms; G means a group of general formula (II)

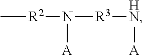

Wherein R2, R3 mutually independently mean a divalent hydrocarbon residue with 1 to 6 carbon atoms, wherein non-adjacent —CH2 units may be replaced by units which are selected from —C(═O)—, —O—, and —S—; A means R5-C(═O)—; R5 means an alkyl residue with 1 to 20 carbon atoms; a means integral values of 100 to 1500; and b means integral values of at least 1.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that particularly advantageous results are achieved if specific silicone(s) and conditioning agents are incorporated into hair treatment agents.

The present invention firstly provides a cosmetic composition containing in a cosmetically acceptable medium

(a) at least one conditioning agent, selected from synthetic oils, mineral oils, plant oils, fluorinated or perfluorinated oils, natural or synthetic waxes, compounds of the ceramide type, carboxylic acid esters, silicones differing from the silicones of formulae (I), anionic polymers, nonionic polymers, cationic polymers, amphoteric polymers, cationic proteins, cationic protein hydrolysates, cationic interfacially active substances and mixtures of these various compounds, and
(b) at least one hydroxy-terminated organopolysiloxane of general formula (I),

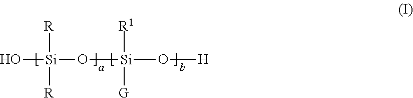

wherein

R means a monovalent unsubstituted or halo-substituted hydrocarbon residue with 1 to 20 carbon atoms,

R¹ means a monovalent unsubstituted or halo-substituted hydrocarbon residue with 1 to 20 carbon atoms, —OR⁴ or —OH,

R⁴ means an alkyl residue with 1 to 6 carbon atoms,

G means a group of general formula (II)

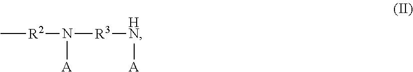

wherein

R², R³ mutually independently mean a divalent hydrocarbon residue with 1 to 6 carbon atoms, wherein non-adjacent —CH₂ units may be replaced by units which are selected from —C(═O)—, —O—, and —S—,

A means R⁵—C(═O)—,

R⁵ means an alkyl residue with 1 to 20 carbon atoms,

a means integral values of 100 to 1500 and

b means integral values of at least 1.

The agent according to the invention is a cosmetic agent. While a distinction was in the past drawn between agents for caring for the human body, “personal hygiene products”, and those for embellishing its appearance, “decorative cosmetics”, these products are today together defined as “cosmetic agents”.

In the light of the general accessibility of cosmetic agents and the use thereof on the human body, there are comprehensive regulatory texts governing consumer protection in Germany and the European Union. The statutory basis in Germany is the Foodstuffs and Consumer Articles Law (LMBG), which in §4 defines cosmetic agents as follows:

“(1) cosmetic agents pursuant to the present law are substances or preparations of substances which are intended to be used externally on a human or in his/her oral cavity for cleaning, caring for or influencing appearance or body odor or for imparting odor impressions, unless they are predominantly intended to alleviate or remedy diseases, illnesses, bodily injury or pathological conditions. §4 LMBG has not hitherto been brought into line with the EU Cosmetics Directive which ascribes six functions (cleaning, perfuming, changing appearance, influencing body odor, protecting and keeping in good condition) to cosmetics.
(2) Substances or preparations of substances for cleaning or caring for dental prostheses are equivalent to cosmetic agents.
(3) Substances or preparations of substances which are intended for influencing body shape are not deemed to be cosmetic agents.”

Depending on the area of application, it is thus possible to differentiate between a wide range of cosmetic agents, for example for skin conditioning (bath preparations, skin washing and cleaning agents, skin care agents, eye cosmetics, lip care agents, nail care agents, intimate hygiene agents, foot care agents), those with specific action (light protection agents, skin-tanning agents, depigmenting agents, deodorants, antiperspirants, depilatory agents, shaving agents, scents), those for dental and oral care (dental and oral care agents, denture care agents, prosthesis adhesives) and those for hair care (shampoos, hair care agents, hair setting agents, hair deforming agents, color-modifying agents).

Cosmetic agents which are preferred according to the invention are selected from the group of shower gels, shower foams, tooth cleaning agents, mouthwashes, shampoos, hair conditioners, conditioning shampoos, hair sprays, hair rinses, hair masks, hair packs, hair tonics, permanent wave fixing solutions, hair coloring shampoos, hair coloring agents, hair strengtheners, hair setting agents, hair styling preparations, blow drying lotions, mousse setting preparations, hair gels, hair waxes or combinations thereof.

More preferred cosmetic agents according to the invention serve to treat keratinic fibers and are thus hair treatment agents. Hair treatment agents for the purposes of the present invention are for example shampoos, hair conditioners, conditioning shampoos, hair sprays, hair rinses, hair masks, hair packs, hair tonics, permanent wave fixing solutions, hair coloring shampoos, hair coloring agents, hair strengtheners, hair setting agents, hair styling preparations, blow drying lotions, mousse setting preparations, hair gels, hair waxes or combinations thereof. In the light of the fact that men often shun using a number of different agents and/or a number of application steps, agents according to the invention are preferably those agents which a man uses in any event. Preferred agents according to the invention are therefore for example shampoos, conditioning agents or hair tonics.

The compositions of the invention have improved cosmetic properties (on hair for example lightness, softness, detanglability, natural feel and high-volume hairstyle, brightness) and the effects are moreover persistent and lasting. In particular, said effects are resistant to many shampoos.

Furthermore, when applied onto the skin (for example by a foam bath or shower gel), the compositions of the invention give rise to improved softness of the skin.

The agents according to the invention contain as first essential ingredient at least one hydroxy-terminated organopolysiloxane of general formula (I),

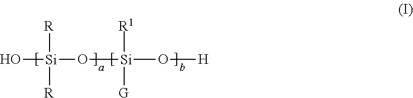

wherein

R means a monovalent unsubstituted or halo-substituted hydrocarbon residue with 1 to 20 carbon atoms,

R¹ means a residue R, —OR⁴ or —OH,

R⁴ means an alkyl residue with 1 to 6 carbon atoms,

G means a group of general formula (II)

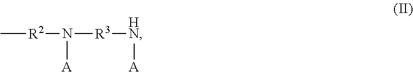

wherein

R², R³ mutually independently mean a divalent hydrocarbon residue with 1 to 6 carbon atoms, wherein non-adjacent —CH₂ units may be replaced by units which are selected from —C(═O)—, —O—, and —S—,

A means R⁵—C(═O)—,

R⁵ means an alkyl residue with 1 to 20 carbon atoms,

a means integral values of 100 to 1500 and

b means integral values of at least 1.

The individual siloxane units a and b in formula (I) and all the following formulae may be present as block copolymers or randomly distributed in each molecule; they are preferably randomly distributed.

As a result of the definitions for G and A, agents according to the invention contain at least one hydroxy-terminated organopolysiloxane of general formula (Ia):

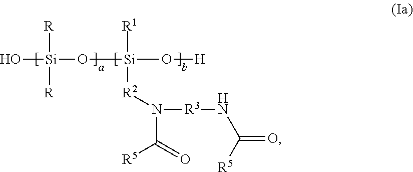

in which R, R¹, R², R³, R⁵, a and b are as defined above.

The residue R¹ may denote a monovalent unsubstituted or halo-substituted hydrocarbon residue with 1 to 20 carbon atoms, an OH group or a residue OR⁴, wherein R⁴ in turn denotes an alkyl residue with 1 to 6 carbon atoms. Preferred residues R¹ are methyl, ethyl, n-propyl, isopropyl, n-butyl, isobutyl, sec-butyl, tert-butyl, n-pentyl, n-hexyl, hydroxy, methoxy, ethoxy, n-propoxy and iso-propoxy residues.

More preferred agents according to the invention contain at least one hydroxy-terminated organopolysiloxane of general formula (Ia-1) and/or (Ia-2) and/or (Ia-3) and/or (Ia-4) and/or (Ia-5) and/or (Ia-6) and/or (Ia-7) and/or (Ia-8) and/or (Ia-9) as shown below:

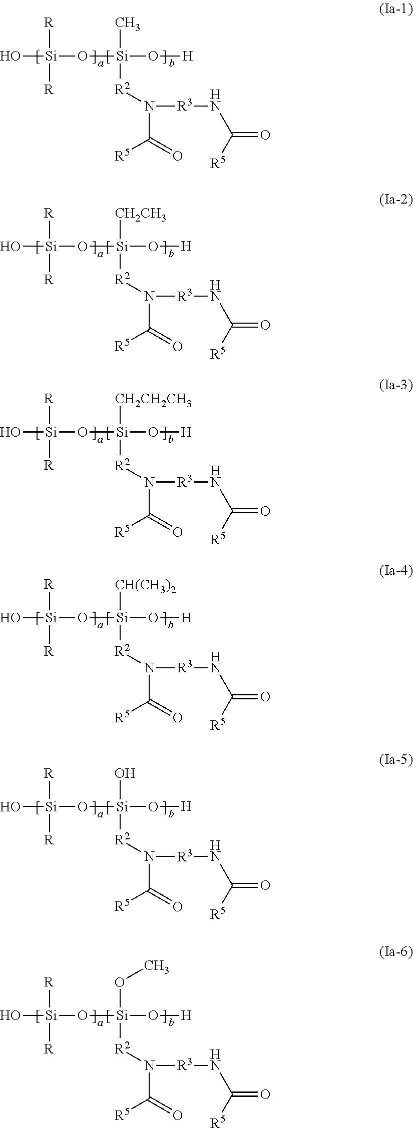

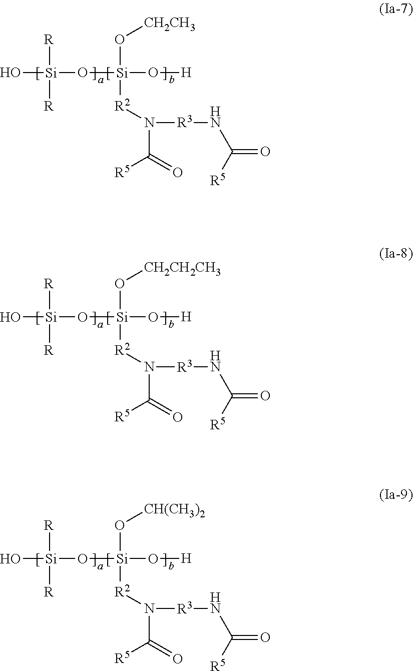

In more preferred cosmetic compositions according to the invention, R is selected from methyl, ethyl, n-propyl, isopropyl, n-butyl, isobutyl, sec-butyl, tert-butyl, n-pentyl, n-hexyl, phenyl residues, wherein R extremely preferably denotes methyl, ethyl or phenyl.

Where R=methyl, formula (Ia) becomes formula (Ib), where R=ethyl it becomes formula (Ic), where R=phenyl it becomes formula (Id), such that more preferred agents according to the invention contain at least one hydroxy-terminated organopolysiloxane of the general formulae (Ib-1) and/or (Ib-2) and/or (Ib-3) and/or (Ib-4) and/or (Ib-5) and/or (Ib-6) and/or (Ib-7) and/or (Ib-8) and/or (Ib-9) and/or (Ic-1) and/or (Ic-2) and/or (Ic-3) and/or (Ic-4) and/or (Ic-5) and/or (Ic-6) and/or (Ic-7) and/or (Ic-8) and/or (Ic-9) and/or (Id-1) and/or (Id-2) and/or (Id-3) and/or (Id-4) and/or (Id-5) and/or (Id-6) and/or (Id-7) and/or (Id-8) and/or (Id-9), each shown below:

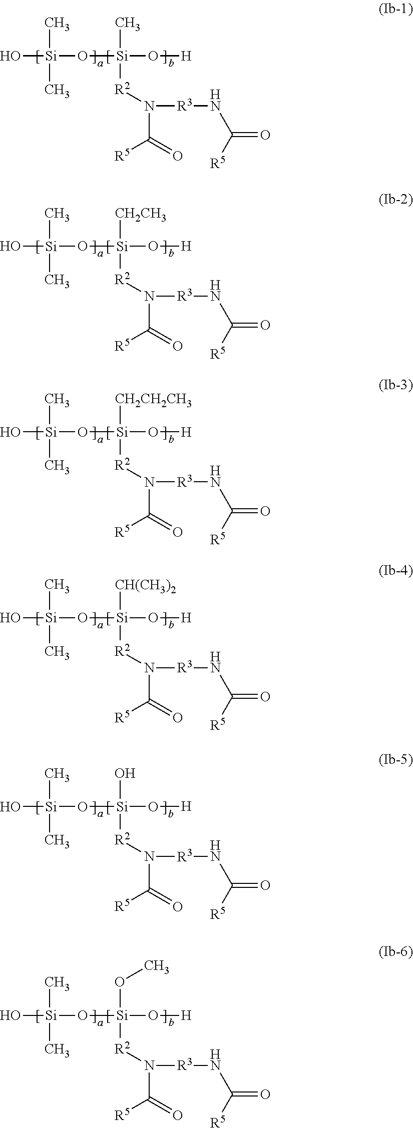

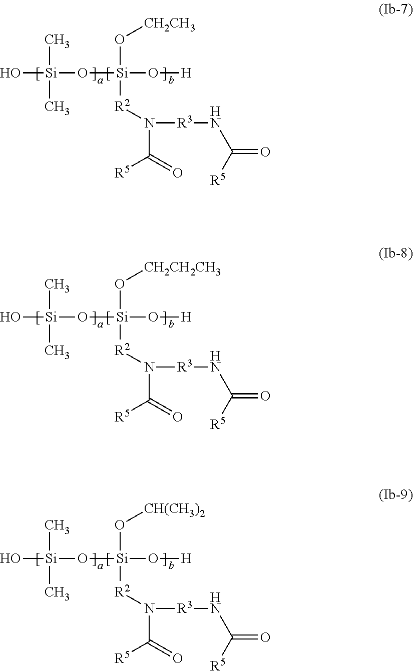

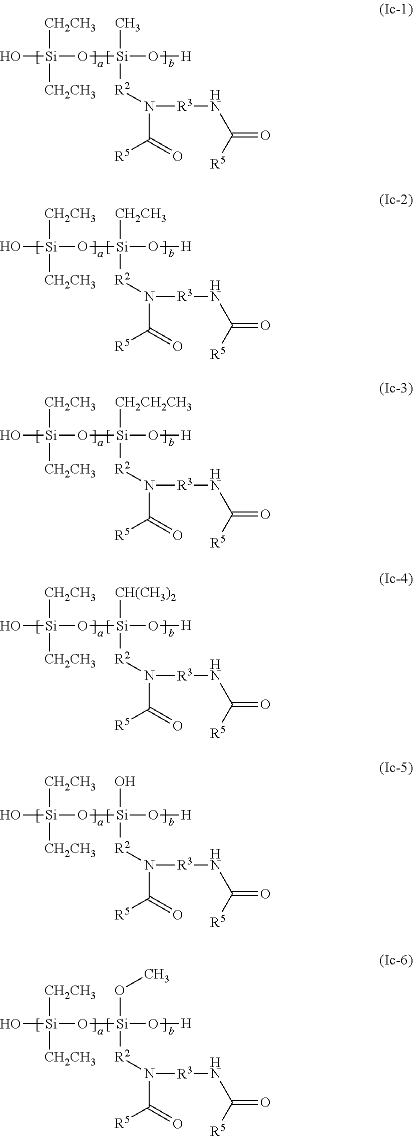

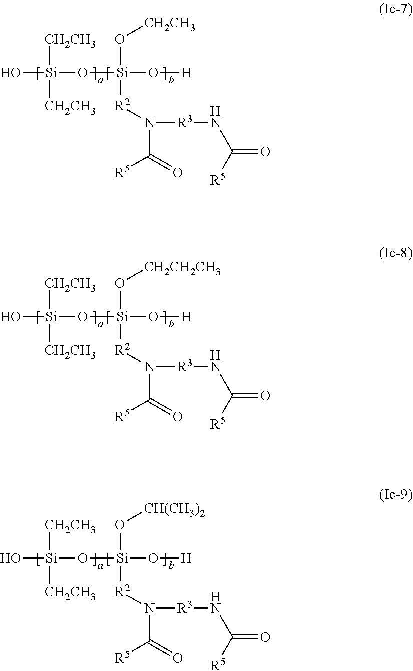

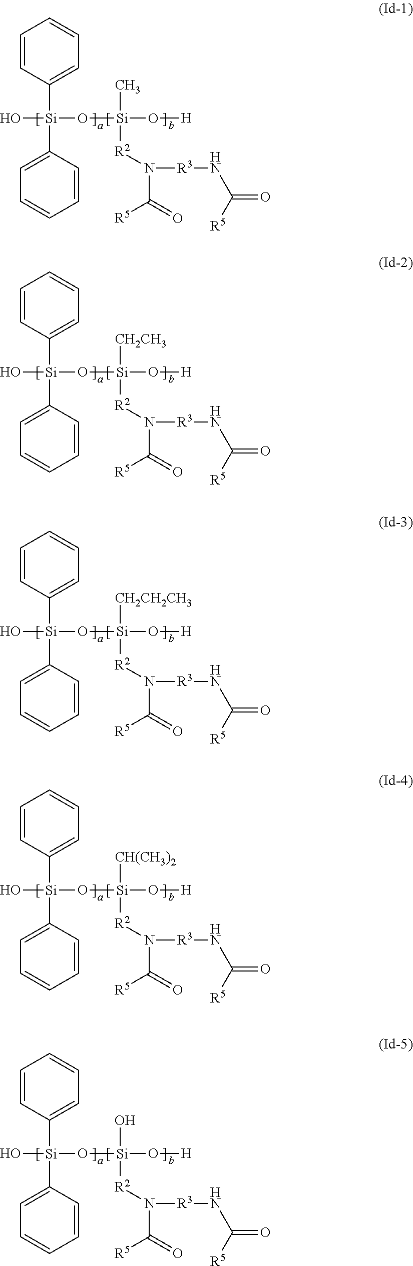

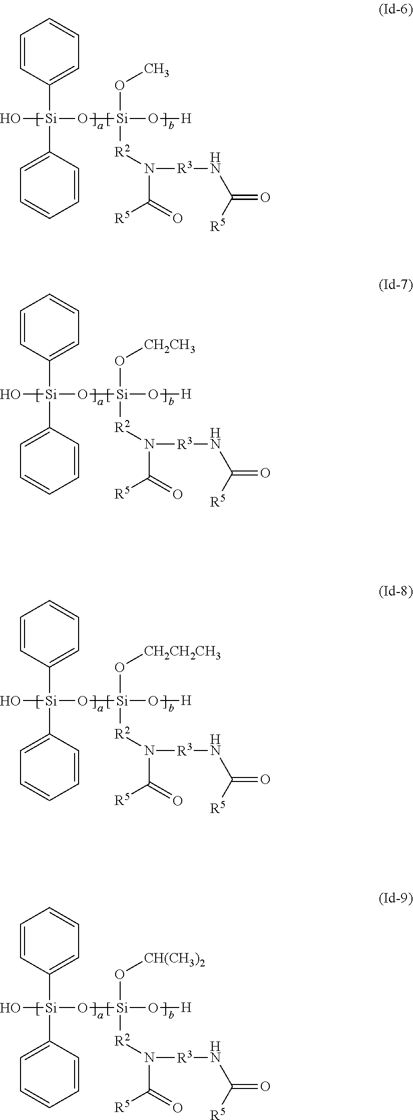

Preferred cosmetic compositions according to the invention contain at least one hydroxy-terminated organopolysiloxane of general formula (I), in which the residues R², R³ are mutually independently selected from ethylene, n-propyl, iso-butylene or n-butylene residues.

R² particularly preferably denotes an n-propylene residue and R³ simultaneously denotes an ethylene residue, such that the grouping G is preferably a grouping

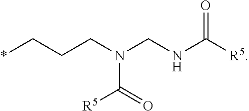

Particularly preferred agents according to the invention therefore contain at least one hydroxy-terminated organopolysiloxane of general formula (Ie-1) and/or (Ie-2) and/or (Ie-3) and/or (Ie-4) and/or (Ie-5) and/or (Ie-6) and/or (Ie-7) and/or (Ie-8) and/or (Ie-9), shown below:

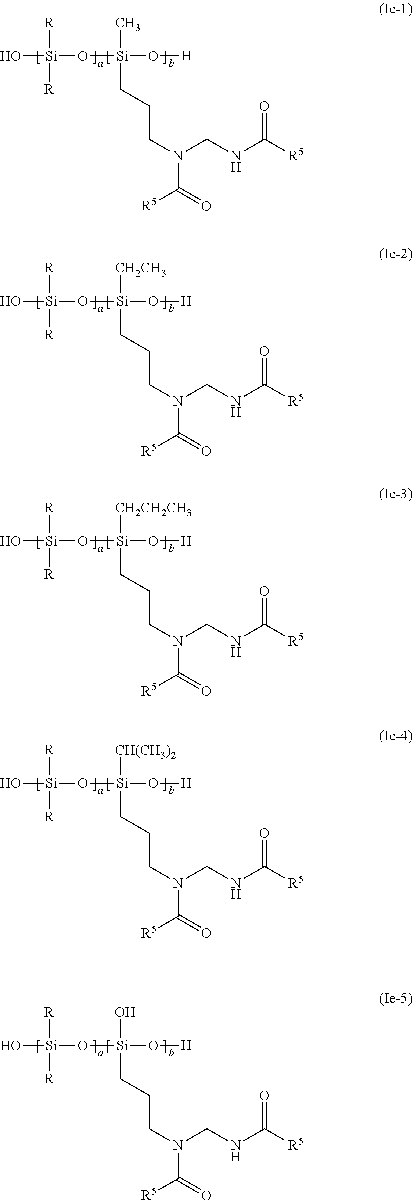

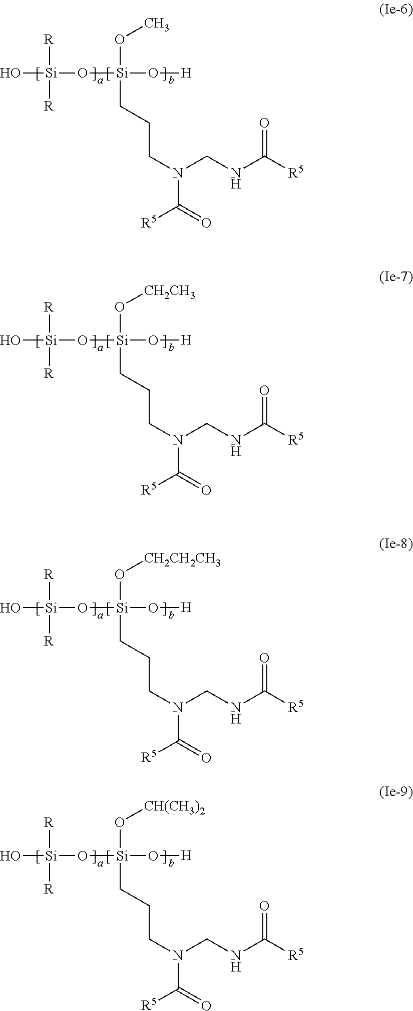

Wherein R groups are as disclosed on pages 14 and 15 of the priority document, and wherein particularly preferred instances of R denote methyl, ethyl or phenyl residues.

Cosmetic compositions according to the invention which are particularly preferred are characterized in that they contain at least one hydroxy-terminated organopolysiloxane of general formula (I), in which R⁵ is selected from methyl, ethyl, n-propyl, isopropyl, n-butyl, isobutyl, sec-butyl, tert-butyl, n-pentyl, n-hexyl residues, wherein R⁵ extremely preferably denotes methyl, ethyl, n-propyl or isopropyl.

Where R⁵=methyl, formula (Ie) becomes formula (If), where R⁵=ethyl it becomes formula (Ig), where R⁵=n-propyl it becomes formula (Ih), where R⁵=isopropyl it becomes formula (II), such that more preferred agents according to the invention contain at least one hydroxy-terminated organopolysiloxane of general formula (If-4) and/or (If-5) and/or (If-6) and/or (If-7) and/or (If-8) and/or (If-9) and/or (Ig-1) and/or (Ig-2) and/or (Ig-3) and/or (Ig-4) and/or (Ig-5) and/or (Ig-6) and/or (Ig-7) and/or (Ig-8) and/or (Ig-9) and/or (Ih-1) and/or (Ih-2) and/or (Ih-3) and/or (Ih-4) and/or (Ih-5) and/or (Ih-6) and/or (Ih-7) and/or (Ih-8) and/or (Ih-9) and/or (Ii-1) and/or (Ii-2) and/or (Ii-3) and/or (Ii-4) and/or (Ii-5) and/or (Ii-6) and/or (Ii-7) and/or (Ii-8) and/or (Ii-9). These formulas are set forth below:

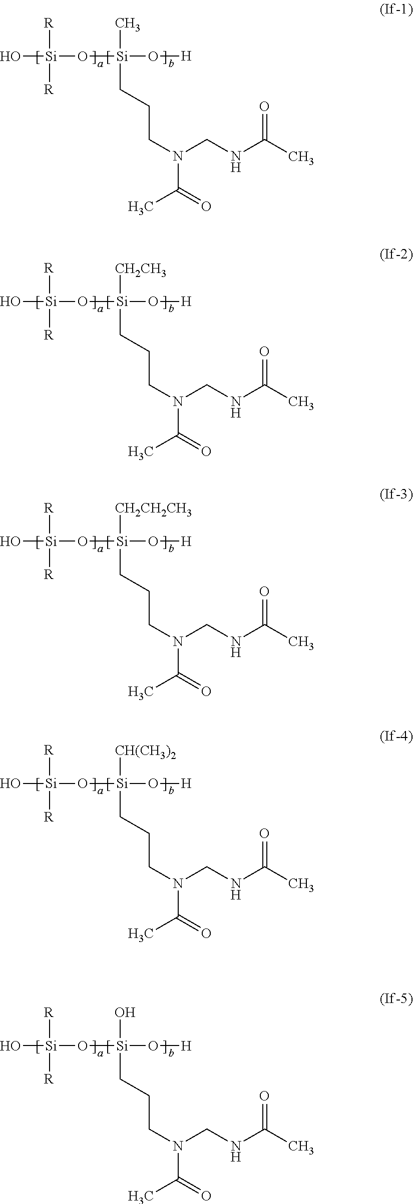

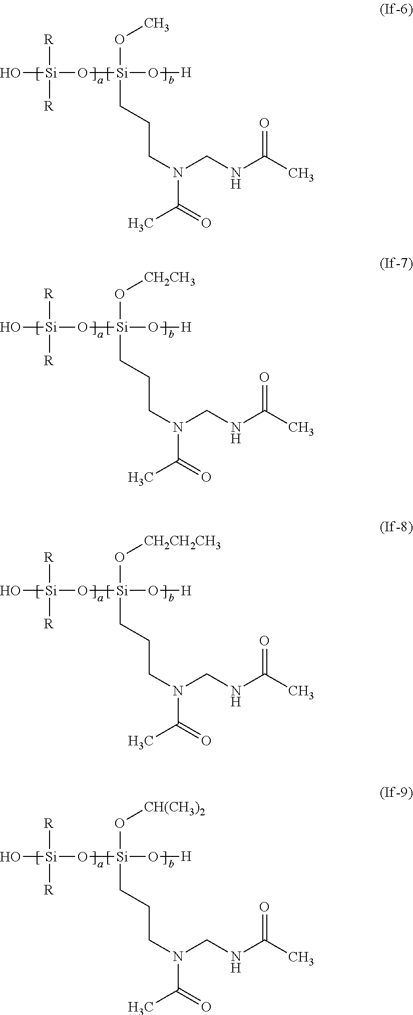

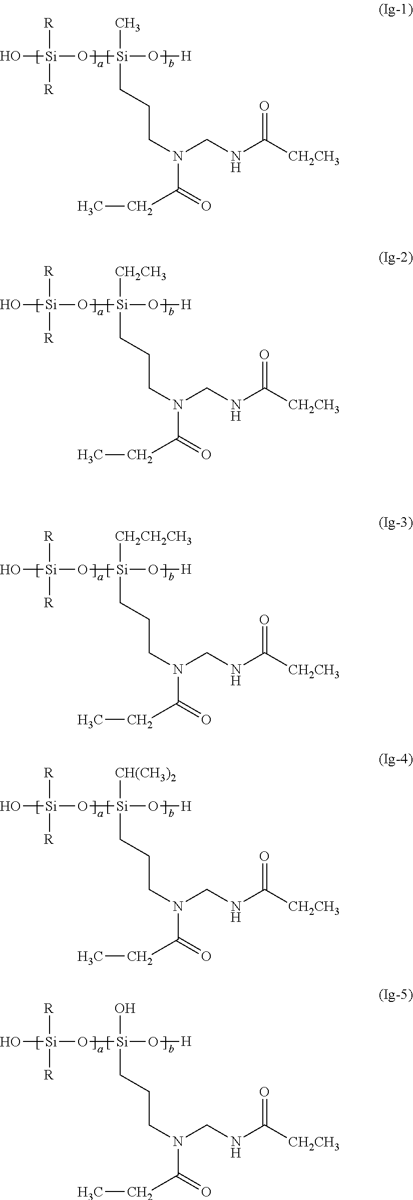

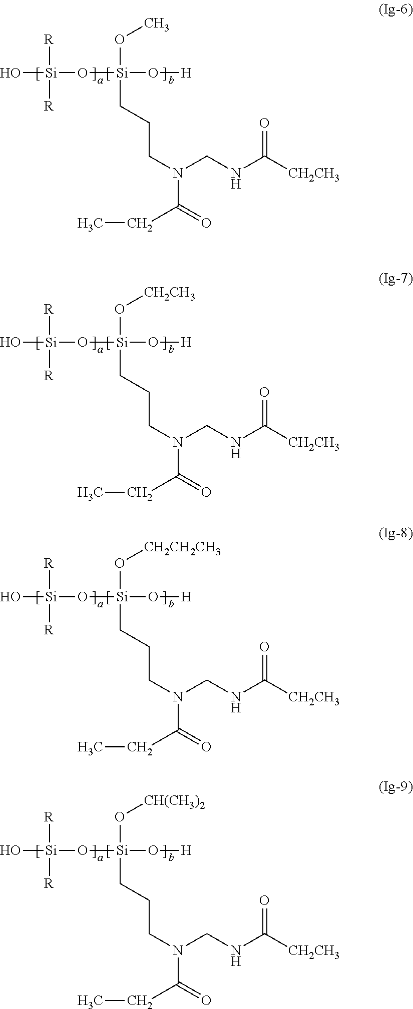

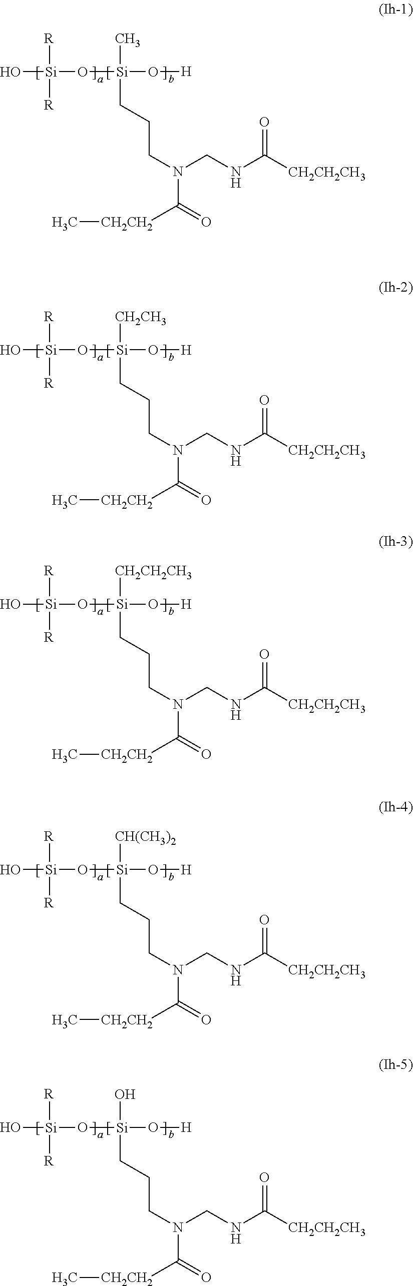

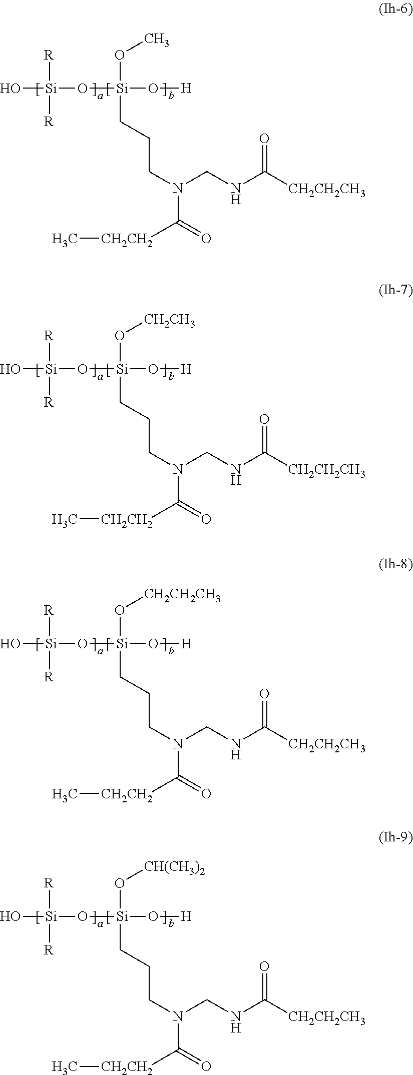

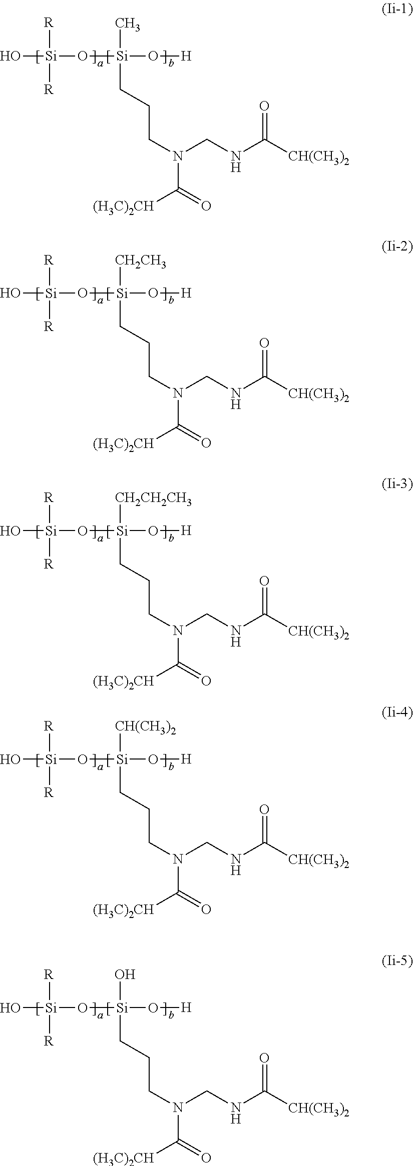

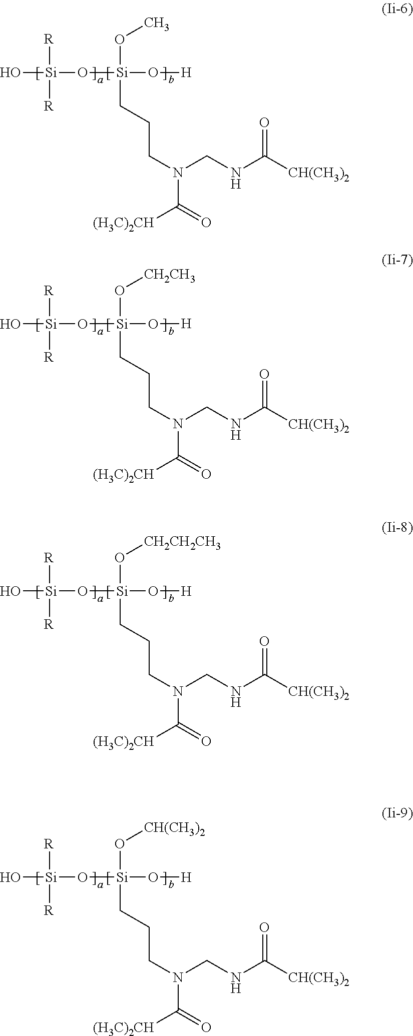

In all the above formulae, those representatives in which R denotes a methyl residue are more preferred.

In all the above formulae, the index a denotes integral values of 100 to 1500. Preferably, a means integral values of at least 200, in particular at least 500 and at most 1300, more preferably at most 1100 and in particular at most 900. Agents according to the invention in which a assumes values of 220 to 910 are more preferred.

In all the above formulae, the index b denotes integral values of at least 1. Preferably, b means integral values of at most 100, in particular at most 50, preferably at most 10 and in particular at most 5. Agents according to the invention in which b assumes the values 1, 2, 3, 4 or 5 are more preferred.

Preferably, a and b are selected such that the organopolysiloxane of formula (I) has, under standard conditions (20° C., 1013.25 mbar), a viscosity (Brookfield RTV, spindle 4, 20 rpm) of at least 100 mPa·s, preferably at least 1000 mPa·s, further preferably at least 5000 mPa·s and in particular at least 15,000 mPa·s. The viscosity preferably amounts to a maximum of 500,000 mPa·s, preferably at most 200,000 mPa·s, more preferably a maximum of 100,000 mPa·s and in particular at most 60,000 mPa·s. Agents according to the invention in which the organopolysiloxane of formula (I) has a viscosity of 17,000 to 55,000 mPa·s are more preferred.

The amine value of the organopolysiloxane of formula (I) preferably amounts to at least 0.001 mmol/g, more preferably at least 0.01 mmol/g. The amine value is more preferably at most 5 mmol/g, further preferably at most 1 mmol/g, still further preferably at most 0.1 mmol/g and in particular a maximum of 0.05 mmol/g. Agents according to the invention in which the organopolysiloxane of formula (I) has amine values of 0.015 to 0.045 mmol/g are more preferred.

Depending on the intended application of the agents according to the invention, the organopolysiloxane(s) of formula (I) may be used in varying quantities. Preferred cosmetic agents according to the invention are characterized in that, relative to the weight thereof, they contain 0.00001 to 10 wt. %, preferably 0.0001 to 7.5 wt. %, further preferably 0.001 to 5 wt. %, further preferably 0.01 to 3 wt. % and in particular 0.1 to 1 wt. % of organopolysiloxane(s) of formula (I).

It has been found that the action of the silicones used according to the invention may be still further increased if specific nonionic components are likewise used in the agents according to the invention. These nonionic components furthermore have positive effects on the storage stability of the agents according to the invention. Nonionic components which are particularly suitable here are ethoxylates of decanol, undecanol, dodecanol, tridecanol etc. Ethoxylated tridecanols have proven particularly suitable and are more preferentially incorporated into the agents according to the invention. Cosmetic compositions which are more preferred according to the invention contain, relative to the weight thereof, 0.00001 to 5 wt. %, preferably 0.0001 to 3.5 wt. %, more preferably 0.001 to 2 wt. %, further preferably 0.01 to 1 wt. % and in particular 0.1 to 0.5 wt. % of branched, ethoxylated tridecanol (INCI name. Trideceth-5) or α-iso-tridecyl-ω-hydroxy polyglycol ether (INCI name: Trideceth-10) or mixtures thereof.

Organopolysiloxane(s) of formula (I) which are preferred according to the invention have both hydroxyl and alkoxy groups. Cosmetic compositions which are more preferred according to the invention contain organopolysiloxane(s) of formula (I) in which the hydroxy/alkoxy molar ratio is in the range from 0.2:1 to 0.4:1, preferably in the range from 1:0.8 to 1:1.1. The average molecular weight of the organopolysiloxane(s) of formula (I) preferably amounts to from 2,000 to 200,000 and still more preferably from 5,000 to 100,000, in particular 10,000 to 50,000 dalton. Cosmetic compositions in which the weight-average molar mass of the organopolysiloxane(s) of formula (I) present therein is in the range from 2,000 to 1,000,000 gmol⁻¹, preferably in the range from 5,000 to 200,000 gmol⁻¹, are preferred.

The average molecular weights of amino-substituted silicones may for example be measured by gel permeation chromatography (GPC) at room temperature in polystyrene. Columns which may be selected are μ-Styragel columns, with THF as eluent and a flow rate of 1 ml/min. Detection preferably proceeds by refractometry and UV meter.

The organopolysiloxane(s) of formula (I) are preferably used as an oil-in-water emulsion. The oil-in-water emulsion may contain one or more surfactants. The surfactants may be of any desired kind, preferably cationic and/or nonionic. The number-average size of the silicone droplets in the emulsion is preferably between 3 nm and 500 nm, more preferably between 5 nm and 60 nm (inclusive) and in particular between 10 nm and 50 nm (inclusive).

Cosmetic compositions according to the invention in which the organopolysiloxane(s) of formula (I) are present in the form of an oil-in-water emulsion, in which the number-average size of the silicone particles in the emulsion is in the range from 3 to 500 nm, preferably in the range from 5 to 60 nm, are preferred according to the invention.

A further essential component of the compositions according to the invention is a conditioning agent selected from synthetic oils, mineral oils, plant oils, fluorinated or perfluorinated oils, natural or synthetic waxes, compounds of the ceramide type, carboxylic acid esters, silicones differing from the silicones of formulae (I), anionic polymers, nonionic polymers, cationic polymers, amphoteric polymers, cationic proteins, cationic protein hydrolysates, cationic interfacially active substances and mixtures of these various compounds.

For the purposes of the present invention, the term “conditioning agent” means any compound which is capable of improving at least one cosmetic property of keratinic material such as hair, for example softness, silkiness, handle, detanglability or static chargeability. The at least one conditioning agent may be water-soluble or water-insoluble.

Water-insoluble conditioning agents may be solid, liquid or pasty at 25° C. and 1013 mbar and may assume the form of oils, waxes, resins or gums.

Water-insoluble conditioning agents may also assume disperse form preferably having a number-average particle or droplet size of 2 nm to 100 μm, preferably of 30 nm to 20 μm. The number-average particle size is here determined using a granulometer. Water-insoluble conditioning agents dissolve in water at 25° C. to an extent of less than 0.1 wt. %, i.e. they do not form macroscopically isotropic, transparent solutions under these conditions.

Synthetic oils, for example polyolefins, in particular poly-alpha-olefins may be selected from: poly-α-olefins of the hydrogenated or non-hydrogenated polybutene type or of the hydrogenated or non-hydrogenated polyisobutene type. Isobutylene oligomers with molar masses of less than 1000 and mixtures thereof, with polyisobutylene with molar masses of greater than 1000, for example of 1000 to 15000 may preferably be used.

Corresponding commercial products are for example Permethyl® 99 A, 101 A, 102 A, 104 A (n=16) and 106 A (n=38) from Presperse Inc., or the products Arlamol® HD (n=3) from ICI (wherein n denotes the degree of polymerization).

Poly-α-olefins of the hydrogenated or non-hydrogenated polydecene type which are distributed under the names Ethylflo® (Ethyl Corp.) and Arlamol® PAO (ICI) may also be used. Cosmetic compositions which are preferred according to the invention are characterized in that the synthetic oil(s) are polyolefins of the hydrogenated or non-hydrogenated polybutene type or of the hydrogenated or non-hydrogenated polydecene type.

Cosmetic oils may particularly preferentially be used as conditioning agents. These oil bodies preferably have a melting point of less than 50° C., more preferably less than 45° C., particularly preferably less than 40° C., highly preferably less than 35° C. and most preferably the cosmetic oils are flowable at a temperature of less than 30° C. These oils are defined and described in greater detail below.

Natural and synthetic cosmetic oils include, for example:

plant oils. Examples of such oils are sunflower oil, olive oil, soy oil, rapeseed oil, almond oil, jojoba oil, orange oil, wheat germ oil, peach stone oil and the liquid fractions of coconut oil. However, other triglyceride oils such as the liquid fractions of beef fat together with synthetic triglyceride oils are also suitable.

liquid paraffin oils, isoparaffin oils and synthetic hydrocarbons and di-n-alkyl ethers with a total of between 12 and 36 C atoms, in particular 12 to 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and di-tert.-butyl ether, di-iso-pentyl ether, di-3-ethyldecyl ether, tert.-butyl-n-octyl ether, iso-pentyl-n-octyl ether and 2-methylpentyl-n-octyl ether. The compounds 1,3-di-(2-ethylhexyl)cyclohexane (Cetiol® S) and di-n-octyl ether (Cetiol®OE) available as commercial products may be preferred.

ester oils. Ester oils should be taken to mean the esters of C₆-C₃₀ fatty acids with C₂-C₃₀ fatty alcohols. The monoesters of fatty acids with alcohols with 2 to 24 C atoms are preferred. Examples of fatty acid moieties used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof, which are obtained, for example, on pressure splitting of natural fats and oils, on oxidation of aldehydes from Roelen's oxo synthesis or the dimerization of unsaturated fatty acids. Examples of fatty alcohol moieties in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof, which are obtained, for example, on high pressure hydrogenation of technical methyl esters based on fats and oils or aldehydes from Roelen's oxo synthesis and as a monomer fraction on the dimerization of unsaturated fatty alcohols. More preferred substances according to the invention are isopropyl myristate (Rilanit® IPM), isononanoic acid C₁₆₋₁₈ alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® mm), cetearyl isononanoate (Cetiol® SN), oleic acid decyl ester (Cetiol® V).

dicarboxylic acid esters such as di-n-butyl adipate, di-(2-ethylhexyl) adipate, di-(2-ethylhexyl) succinate and diisotridecyl acelate and diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate,

symmetrical, asymmetrical or cyclic esters of carbonic acid with fatty alcohols, for example described in DE-OS 197 56 454, glycerol carbonate or dicaprylyl carbonate (Cetiol® CC),

trifatty acid esters of saturated and/or unsaturated linear and/or branched fatty acids with glycerol,

fatty acid partial glycerides, i.e. monoglycerides, diglycerides and the technical mixtures thereof. When using technical products, small quantities of triglycerides may still be present therein, depending on the production method. Partial glycerides preferably comply with formula (D4-I),

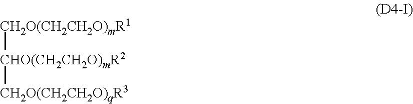

in which R¹, R² and R³ mutually independently denote hydrogen or a linear or branched, saturated and/or unsaturated acyl residue with 6 to 22, preferably 12 to 18, carbon atoms, with the proviso that at least one of these groups denotes an acyl residue and at least one of these groups denotes hydrogen. The sum (m+n+q) denotes 0 or numbers from 1 to 100, preferably 0 or 5 to 25. Preferably, R¹ denotes an acyl residue and R² and R³ denote hydrogen and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof. Preferably, oleic acid monoglycerides are used.

Natural oils which may be considered are for example amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, maize germ oil, almond oil, marula oil, evening primrose oil, olive oil, palm oil, rapeseed oil, rice oil, sea buckthorn fruit oil, sea buckthorn seed oil, sesame oil, shea butter, soy oil, sunflower oil, grapeseed oil, walnut oil or wild rose oil.

It goes without saying that the teaching according to the invention also includes its being possible for at least two of the natural oils listed the table above to be mixed with one another. In this case, the natural oils can be selected such that the sum of the fatty acids palmitic acid, stearic acid and linoleic acid constitutes at least 50 wt. % of the sum of the fatty acids. Preferred mixtures of the natural oils are amaranth seed oil with at least one sea buckthorn oil, amaranth seed oil with shea butter, amaranth seed oil with camelina oil, amaranth seed oil with olive oil, amaranth seed oil with macadamia nut oil, olive oil with at least one sea buckthorn oil, olive oil with camelina oil, olive oil with shea butter, macadamia nut oil and at least one sea buckthorn oil, macadamia nut oil with shea butter. More than at most three of the natural oils should, however, not be mixed with one another. Argan oil is one of the more preferred natural oils.

Preferred cosmetic compositions according to the invention are characterized in that the plant oil(s) are selected from sunflower oil, corn oil, soy oil, avocado oil, jojoba oil, pumpkin seed oil, grapeseed oil, sesame oil, hazelnut oil, fish oils, glycerol tricaprocaprylate or plant or animal oils of formula R⁹COOR¹⁰, in which R⁹ means the residue of a higher fatty acid with 7 to 29 carbon atoms and R¹⁰ means a linear or branched hydrocarbon chain with 3 to 30 carbon atoms, natural or synthetic essential oils.

Natural or synthetic waxes (Fatwax) which may be used according to the invention are solid paraffins or isoparaffins, carnauba waxes, beeswaxes, candelilla waxes, ozokerites, ceresin, spermaceti, sunflower wax, fruit waxes such as for example apple wax or citrus wax, PE or PP microwaxes.

Preferred cosmetic compositions according to the invention are characterized in that the wax or waxes are selected from carnauba wax, candelilla wax, Alfa wax, paraffin wax, ozokerite, plant waxes, animal waxes, polyethylene waxes or polyolefin waxes.

The quantity used amounts to 0.1-50 wt. % relative to the entire agent, preferably 0.1-20 wt. % and more preferably 0.1-15 wt. % relative to the entire agent.

Compounds of the ceramide type may for example be selected from natural and synthetic ceramides, glycoceramides, pseudoceramides and neoceramides. Preferred representatives from these groups are 2-N-linoleoylaminooctadecane-1,3-diol, 2-N-oleoylaminooctadecane-1,3-diol, 2-N-palmitoylaminooctadecane-1,3-diol, 2-N-stearoylaminooctadecane-1,3-diol, 2-N-behenoylaminooctadecane-1,3-diol, 2-N-[2-hydroxypalmitoyl]aminooctadecane-1,3-diol, 2-N-stearoylaminooctadecane-1,3,4-triol and such as N-stearoylphytosphingosine, 2-N-palmitoylaminohexadecane-1,3-diol, bis(N-hydroxyethyl-N-cetyl)malonamide, N-(2-hydroxyethyl)-N-(3-cetyloxy-2-hydroxy-propyl)cetylamide, N-docosanoyl-N-methyl-D-glucamine and mixtures of these compounds.

Preferred cosmetic compositions according to the invention are characterized in that the compounds of the ceramide type are selected from 2-N-linoleoylaminooctadecane-1,3-diol, 2-N-oleoylaminooctadecane-1,3-diol, 2-N-palmitoylaminooctadecane-1,3-diol, 2-N-stearoylaminooctadecane-1,3-diol, 2-N-behenoylaminooctadecane-1,3-diol, 2-N-[2-hydroxypalmitoyl]aminooctadecane-1,3-diol, 2-N-stearoylaminooctadecane-1,3,4-triol, 2-N-palmitoylaminohexadecane-1,3-diol, bis(N-hydroxyethyl-N-cetyl)malonamide, N-(2-hydroxyethyl)-N-(3-cetyloxy-2-hydroxypropyl)amide of cetylic acid, N-docosanol-N-methyl-D-glucamine or mixtures these compounds.

Fatty alcohols should be taken to mean primary aliphatic alcohols of formula R¹OH

in which R¹ denotes an aliphatic, linear or branched hydrocarbon residue with 6 to 22 carbon atoms and 0 and/or 1, 2 or 3 double bonds. Typical examples are caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof, which are obtained, for example, on high pressure hydrogenation of technical methyl esters based on fats and oils or aldehydes from Roelen's oxo synthesis and as a monomer fraction on the dimerization of unsaturated fatty alcohols. Preferred technical fatty alcohols are those with 12 to 18 carbon atoms, such as for example coconut, palm, palm kernel or tallow fatty alcohol. Alkoxylated fatty alcohols with 1 to 15 mol of alkylene oxide, or polyglycerylated compounds with 1 to 6 mol of glycerol may also be used as conditioning agents.

Monocarboxylic acid esters may particularly preferentially also be used as conditioning agents. These are for example selected from linear or branched, saturated or unsaturated aliphatic C₁-C₂₆ monoesters of linear or branched, saturated or unsaturated aliphatic C₁-C₂₆ alcohols, wherein the total number of carbon atoms in the esters is 10 or more.

More preferred conditioning agents are dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate, isocetyl laurate, isocetyl stearate, isodecyl octanoate, isodecyl oleate, isononyl isononanoate, isostearyl palmitate, methylacetyl ricinoleate, myristyl stearate, octyl isononanoate, 2-ethylhexyl isononate, octyl palmitate, octyl pelargonate, octyl stearate, octyldodecyl erucate, oleyl erucate, ethyl and isopropyl palmitate, 2-ethylhexyl palmitate, 2-octyldecyl palmitate, alkyl myristates such as isopropyl-, butyl-, cetyl- and 2-octyldodecyl myristate, hexyl stearate, butyl stearate, isobutyl stearate, dioctyl malate, hexyl laurate and 2-hexyldecyl laurate. C₄-C₂₂ di- or tricarboxylic acid esters of C₁-C₂₋₂ alcohols and mono-, di- or tricarboxylic acid esters of C₂-C₂₆ di-, tri-, tetra- or pentahydroxy alcohols may likewise be used.

The following may here be mentioned diethyl sebacate, diisopropyl sebacate, diisopropyl adipate, di-n-propyl adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate/dicaprate, tridecyl erucate, triisopropyl citrate, triisostearyl citrate, glyceryl trilactate, glyceryl trioctanoate, trioctyldodecyl citrate, and trioleyl citrate.

Some of the above-stated esters are more preferred. Cosmetic compositions according to the invention in which the carboxylic acid esters are selected from ethyl palmitate and isopropyl palmitate, 2-ethylhexyl palmitate, 2-octyldecyl palmitate, alkyl myristates, hexyl stearate, butyl stearate, isobutyl stearate; dioctyl malate, hexyl laurate, 2-hexyldecyl laurate, isononyl isononanoate and cetyl octanoate are preferred according to the invention.

Silicones which differ from the silicones of formula (I) used according to the invention may also be used as conditioning agents.

Preferred agents according to the invention are characterized in that they contain at least one further silicone, preferably a silicone which is selected from:

(i) polyalkylsiloxanes, polyarylsiloxanes, polyalkylarylsiloxanes, which are volatile or non-volatile, straight-chain, branched or cyclic, crosslinked or uncrosslinked;
(ii) polysiloxanes which contain in their general structure one or more organofunctional groups which are selected from:
a) substituted or unsubstituted aminated groups;
b) (per)fluorinated groups;
c) thiol groups;
d) carboxylate groups;
e) hydroxylated groups;
f) alkoxylated groups;
g) acyloxyalkyl groups;
h) amphoteric groups;
i) bisulfite groups;
j) hydroxyacylamino groups;
k) carboxy groups;
l) sulfonic acid groups; and
m) sulfate or thiosulfate groups;
(iii) linear polysiloxane(A)/polyoxyalkylene(B) block copolymers of the type (A-B)ₙ with n>3;
(iv) grafted silicone polymers having an organic parent structure containing no silicone, which polymers consist of an organic main chain which is formed from organic monomers containing no silicone and onto which at least one polysiloxane macromer has been grafted in the chain and optionally onto at least one chain end;
(v) grafted silicone polymers with a polysiloxane parent structure, onto which organic monomers containing no silicone have been grafted, which silicone polymers have a polysiloxane main chain onto which at least one organic macromer has been grafted in the chain and optionally onto at least one of the ends thereof; or mixtures thereof.

More preferred cosmetic compositions according to the invention are characterized in that the silicones which differ from the silicones of formula (I) are non-volatile polyorganosiloxanes which are selected from polyalkylsiloxanes, polyarylsiloxanes, polyalkylarylsiloxanes, silicone gums, silicone resins, polyorganosiloxanes modified with organofunctional groups and mixtures thereof. Particularly preferred cosmetic compositions according to the invention are characterized in that

(a) the polyalkylsiloxanes are selected from:

polydimethylsiloxanes with terminal trimethylsilyl groups;

polydimethylsiloxanes with terminal dimethylsilyl groups;

polyalkyl(C₁₋₂₀)siloxanes;

(b) the polyarylsiloxanes are selected from:

polydimethylmethylphenylsiloxanes, polydimethyldiphenylsiloxanes which assume straight-chain and/or branched form and at 25° C. have a viscosity in the range from 1·10⁻⁵ to 5·10⁻² m²/s;

(c) the silicone gums are selected from polydiorganosiloxanes which have number-average molar masses in the range from 200 000 to 1 000 000 and which are used as such or mixed with a solvent;
(d) the resins are selected from resins which are synthesized from units R³SiO_{1/2}, R²SiO_{2/2}, RSiO_{3/2} and SiO_{4/2}, in which the group R means a hydrocarbon group with 1 to 16 carbon atoms or a phenyl group;
(e) the organo-modified silicones are selected from silicones which bear in the structure thereof one or more organofunctional groups attached via a hydrocarbon group.

Agents which are more preferred according to the invention contain the further silicone(s) preferably in quantities of 0.1 to 10 wt. %, preferably of 0.25 to 7 wt. % and in particular of 0.5 to 5 wt. %, in each case relative to the entire agent.

Preferred silicones are described hereinafter.

More preferred agents according to the invention are characterized in that they contain at least one amino-functional silicone of formula Si-I

(CH₃)₃Si—[O—Si(CH₃)₂]ₓ—O—Si(CH₃)₃ (Si-I),

in which x denotes a number from 0 to 100, preferably from 0 to 50, further preferably from 0 to 20 and in particular from 0 to 10.

According to INCI nomenclature, these silicones are known as Dimethicones. Compounds of the following formulae are preferably used as the silicone of formula Si-I: (CH₃)₃Si—O—Si(CH₃)₃, (CH₃)₃Si—O—(CH₃)₂Si—O—Si(CH₃)₃, (CH₃)₃Si—[O—(CH₃)₂Si]₂—O—Si(CH₃)₃, (CH₃)₃Si—[O—(CH₃)₂Si]₃—O—Si(CH₃)₃, (CH₃)₃Si—[O—(CH₃)₂Si]₄—O—Si(CH₃)₃, (CH₃)₃Si—[O—(CH₃)₂Si]₅—O—Si(CH₃)₃, (CH₃)₃Si—[O—(CH₃)₂Si]₆—O—Si(CH₃)₃, (CH₃)₃Si—[O—(CH₃)₂Si(CH₃)₃, (CH₃)₃Si—[O—(CH₃)₂Si]₁₁—O—Si(CH₃)₃, (CH₃)₃Si[O—(CH₃)₂Si]₉—O—Si(CH₃)₃, (CH₃)₃Si[O—(CH₃)₂Si]₁₀—O—Si(CH₃)₃, (CH₃)₃Si—[O—(CH₃)₂Si]—O—Si(CH₃)₃, (CH₃)₃Si[O—(CH₃)₂Si]₁₂—O—Si(CH₃)₃, (CH₃)₃Si—[O—(CH₃)₂Si]₁₃—O—Si(CH₃)₃, (CH₃)₃Si—[O—(CH₃)₂Si]—₁₄—Si(CH₃)₃, (CH₃)₃Si—[O—(CH₃)₂Si]₅—O—Si(CH₃)₃, (CH₃)₃Si—[O—(CH₃)₂Si]₁₆—O—Si(CH₃)₃, (CH₃)₃Si—[O—(CH₃)₂Si]₁₇—O—Si(CH₃)₃, (CH₃)₃Si[—O—(CH₃)₂Si]₁₈—O—Si(CH₃)₃, (CH₃)₃Si—[O—(CH₃)₂Si]₁₉—O—Si(CH₃)₃, (CH₃)₃Si—[O—(CH₃)₂Si]₂₀—O—Si(CH₃)₃, wherein (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ are more preferred. Mixtures of the above-stated silicones may of course be present in agents according to the invention.

Silicones which are preferably usable according to the invention exhibit viscosities at 20° C. of 0.2 to 2 mm²s⁻¹, wherein silicones having viscosities of 0.5 to 1 mm²s⁻¹ are more preferred. Preferred cosmetic compositions according to the invention are characterized in that the silicones used individually or in combination are selected from the following structures

polydimethylsiloxane,

polydimethylsiloxane/methylvinylsiloxanes,

polydimethylsiloxane/diphenylsiloxane,

polydimethylsiloxane/phenylmethylsiloxane,

polydimethylsiloxane/diphenylsiloxane/methylvinylsiloxane and the following mixtures:

mixtures composed of a chain end-hydroxylated polydimethylsiloxane and a cyclic polydimethylsiloxane,

mixtures composed of a polydimethylsiloxane gum and a cyclic silicone, and

mixtures of polydimethylsiloxanes of various viscosities.

More preferred agents according to the invention contain one or more organo-modified silicones since the applicational properties of the silicones may be adapted still more closely to the intended application by the modification. Cosmetic compositions which are preferred according to the invention are characterized in that the organo-modified silicones are selected from polyorganosiloxanes which contain a) polyethyleneoxy and/or polypropyleneoxy groups, b) substituted or unsubstituted aminated groups, c) thiol groups, d) alkoxylated groups, e) hydroxyalkyl groups, f) acyloxyalkyl groups, g) carboxyalkyl groups, h) 2-hydroxyalkylsulfonate groups, i) 2-hydroxyalkylthiosulfonate groups, j) hydroxyacylamino groups. More preferred agents according to the invention contain one or more amino-functional silicones. Such silicones may, for example, be described by the formula

M(RₐO_{b}SiO_{(4-a-b)/2)x}(R_{c}SiO_{(4-c)/2)y}M

wherein in the above formula R is a hydrocarbon or a hydrocarbon residue with 1 to approx. 6 carbon atoms, Q is a polar residue of general formula —R¹HZ, in which R¹ is a divalent linking group, which is attached to hydrogen and the residue Z, and is composed of carbon and hydrogen atoms, carbon, hydrogen and oxygen atoms or carbon, hydrogen and nitrogen atoms, and Z is an organic, amino-functional residue which contains at least one amino-functional group; “a” assumes values in the range from approx. 0 to approx. 2, “b” assumes values in the range from approx. 1 to approx. 3, “a”+“b” is less than or equal to 3, and “c” is a number in the range from approx. 1 to approx. 3, and x is a number in the range from 1 to approx. 2,000, preferably from approx. 3 to approx. 50 and most preferably from approx. 3 to approx. 25, and y is a number in the range from approx. 20 to approx. 10,000, preferably from approx. 125 to approx. 10,000 and most preferably from approx. 150 to approx. 1,000, and M is a suitable silicone end group, as is known in the prior art, preferably trimethylsiloxy. Non-limiting examples of the residues represented by R include alkyl residues, such as methyl, ethyl, propyl, isopropyl, butyl, isobutyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; R is preferably an alkyl residue, which contains 1 to approx. 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional residue containing at least one functional amino group. A possible formula for Z is NH(CH₂)_{z}NH₂, in which z is 1 or more. Another possible formula for Z is —NH(CH₂)_{z}(CH₂)_{zz}NH, in which both z and zz are mutually independently 1 or more, wherein this structure comprises diamino ring structures, such as piperazinyl. Z is most preferably an —NHCH₂CH₂NH₂ residue. Another possible formula for Z is —N(CH₂)_{z}(CH₂)_{zz}NX₂ or —NX₂, in which each X of X₂ is independently selected from the group consisting of hydrogen and alkyl groups having 1 to 12 carbon atoms, and zz is 0.

Q is most preferably a polar amino-functional residue of formula —CH₂CH₂CH₂NHCH₂CH₂NH₂. In the formulae, “a” assumes values in the range from approx. 0 to approx. 2, “b” assumes values in the range from approx. 2 to approx. 3, “a”+“b” is less than or equal to 3, and “c” is a number in the range from approx. 1 to approx. 3. The molar ratio of the RₐQbSiO_{(4-a-b)/2} units to the R_{c}SiO_{(4-c)/2} units is in the range from approx. 1:2 to 1:65, preferably from approx. 1:5 to approx. 1:65 and most preferably from approx. 1:15 to approx. 1:20. If one or more silicones of the above formula are used, then the various variable substituents in the above formula may be different in the various silicone components which are present in the silicone mixture.

Preferred agents according to the invention are characterized in that they contain an amino-functional silicone of formula (Si-II)

R′ₐG₃₋ₐ-Si(OSiG₂)ₙ—(OSiGbR′_{2-b})ₘ—O—SiG₃₋ₐ—R′ₐ (Si-II),

in which:

G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;

a denotes a number between 0 and 3, in particular 0;

b denotes a number between 0 and 1, in particular 1,

m and n are numbers, the sum of which (m+n) amounts to between 1 and 2000, preferably between 50 and 150, wherein n preferably assumes values from 0 to 1999 and in particular from 49 to 149 and m preferably assumes values from 1 to 2000, in particular from 1 to 10,

R′ is a monovalent residue selected from
-Q-N(R″)—CH₂—CH₂—N(R″)₂
-Q-N(R″)₂
-Q-N⁺(R″)₃A⁻
-Q-N⁺H(R″)₂A⁻
-Q-N⁺H₂(R″)A⁻
-Q-N(R″)—CH₂—CH₂—N⁺R″H₂A⁻,
   wherein each Q denotes a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
   R″ denotes identical or different residues from the group —H, phenyl, benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide or methosulfate.

More preferred agents according to the invention are characterized in that they contain at least one amino-functional silicone of formula (Si-IIa)

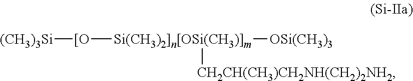

in which m and n are numbers, the sum of which (m+n) amounts to between 1 and 2000, preferably between 50 and 150, wherein n preferably assumes values from 0 to 1999 and in particular from 49 to 149 and m preferably assumes values from 1 to 2000, in particular from 1 to 10.

These silicones are denoted in accordance with the INCI Declaration as Trimethylsilylamodimethicones. Agents according to the invention which are more preferred are also those which contain an amino-functional silicone of formula (Si-IIb)

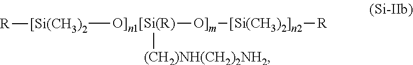

in which R denotes —OH, —O—CH₃ or a —CH₃ group and m, n1 and n2 are numbers the sum of which (m+n1+n2) amounts to between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

These silicones are denoted in accordance with the INCI Declaration as Amodimethicones.

Irrespective of which amino-functional silicones are used, preferred agents according to the invention are those which contain an amino-functional silicone, the amine value of which is above 0.25 meq/g, preferably above 0.3 meq/g and in particular above 0.4 meq/g. The amine value here denotes the milliequivalents of amine per gram of the amino-functional silicone. It can be determined by titration and may also be stated in the unit mg of KOH/g.

Agents which are preferred according to the invention are characterized in that, relative to the weight thereof, they contain 0.01 to 10 wt. %, preferably 0.1 to 8 wt. %, more preferably 0.25 to 7.5 wt. % and in particular 0.5 to 5 wt. % of amino-functional silicone(s).

The cyclic dimethicones designated according to INCI as Cyclomethicones may preferably be used according to the invention. Preferred agents according to the invention are here those which contain at least one silicone of formula Si-III

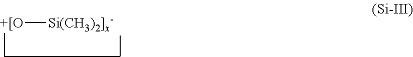

in which x denotes a number from 3 to 200, preferably from 3 to 10, further preferably from 3 to 7 and in particular 3, 4, 5 or 6.

Cyclic silicones with 3 to 7, preferably 4 to 5 Si-atoms are for example octamethylcyclotetrasiloxane, obtainable as “Volatile Silicone 7207” (Union Carbide) or “Silbione 70045 V 2” (Rhodia Chimie), decamethylcyclopentasiloxane, obtainable as “Volatile Silicone 7158” (Union Carbide) and “Silbione 70045 V 5” (Rhodia Chimie) and mixtures thereof.

Cyclocopolymers of the dimethylsiloxane/methylalkylsiloxane type are for example “Volatile Silicone FZ 3109” (Union Carbide), with the structure:

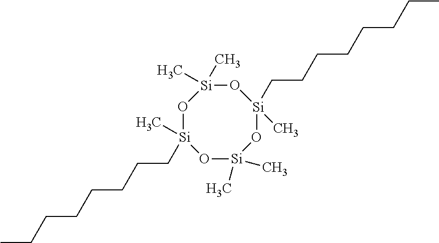 [SMILES: CCCCCCCC[Si]1(C)O[Si](C)(C)O[Si](C)(CCCCCCCC)O[Si](C)(C)O1]

It is also possible to use mixtures of cyclic silicones with organosilicone compounds such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane.

The above-described silicones have a backbone which is made up of —Si—O—Si units. These Si—O—Si units may, of course, also be interrupted by carbon chains Corresponding molecules are obtainable by chain extension reactions and are preferably used in the form of silicone-in-water emulsions.

Agents which are likewise preferred according to the invention are characterized in that they contain at least one silicone of formula S1—IV

R³Si—[O—SiR²]ₓ—(CH₂)ₙ—[O—SiR²]_{y}—O—SiR³ (Si-IV),

in which R denotes identical or different residues from the group —H, phenyl, benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, further preferably from 0 to 7 and in particular 0, 1, 2, 3, 4, 5 or 6, and n denotes a number from 0 to 10, preferably from 1 to 8 and in particular 2, 3, 4, 5, 6.

The silicones are preferably water-soluble. Preferred agents according to the invention are characterized in that they contain at least one water-soluble silicone.

Particularly preferred cosmetic compositions are characterized in that the silicones are selected from polyalkylsiloxanes with terminal trimethylsilyl groups, polyalkylsiloxanes with terminal dimethylsilanol groups, polyalkylarylsiloxanes, mixtures of two PDMS which are formed from a gum and an oil of different viscosities, mixtures of organosiloxanes and cyclic silicones and organopolysiloxane resins.

Trade names of silicones preferably used as conditioning agents are for example

decamethyltetrasiloxane “SH 200” (Toray Silicone)

Silbione® oils of the 47 and 70 047 series

Mirasil® oils (Rhodia Chimie) such as 70 047 V 500 000

oils of the 200 series (Dow Corning), such as DC200 with a viscosity of 60 000 cSt (mm²/s)

Viscasil® oils (General Electric)

oils of the SF series (SF 96, SF 18, SF 1023, SF 1154, SF 1250 and SF 1265) from General Electric

dimethiconols such as oils of the 48 series (Rhodia Chimie)

Abil® Wax 9800 and 9801 (Goldschmidt)

Silbione oils of the 70 641 series (Rhodia Chimie)

oils of the Rhodorsil 70 633 and 763 series (Rhodia Chimie)

Dow Corning 556 Cosmetic Grade Fluid (Dow Corning)

silicones of the PK series (Bayer), such as for example PK20

silicones of the PN and PH series (Bayer), such as for example PN1000 and PH1000

Q2 1401 (Dow Corning)

SF 1214 Silicone Fluid (General Electric)

SF 1236 (General Electric)

“Dow Corning 593” or “Silicone Fluid SS 4230 and SS 4267” (General Electric)

X22-4914, X21-5034 and X21-5037 (Shin-Etsu)

DC 1248 and Q2 5200 (Dow Corning)

GP 4 Silicone Fluid and GP 7100 (Genesee)

Q2 8220 and Dow Corning 929 or 939 (Dow Corning)

GP 72 A and GP 71 (Genesee)

Silicone Copolymer F-755 (SWS Silicones) and Abil Wax 2428, 2434 and 2440 (Goldschmidt)

X-22-3701 E (Shin-Etsu)

Abil 5201 and Abil 5255 (Goldschmidt)

Q2-8413 (Dow Corning).

Water-soluble conditioning agents, preferably from the groups of anionic polymers, nonionic polymers, cationic polymers, amphoteric polymers, cationic proteins and protein hydrolysates, cationic surfactants and mixtures of these substances may also be used as conditioning agents.

The anionic polymers which can support the action of the active ingredient according to the invention are anionic polymers which comprise carboxylate and/or sulfonate groups. Examples of anionic monomers of which such polymers may consist are acrylic acid, methacrylic acid, crotonic acid, maleic anhydride and 2-acrylamido-2-methylpropanesulfonic acid. In this case, the acidic groups may be present wholly or in part as a sodium, potassium, ammonium, mono- or triethanolammonium salt. 2-Acrylamido-2-methylpropanesulfonic acid and acrylic acid are preferred monomers.

Anionic polymers which have proven very particularly effective are those which contain as sole or co-monomer 2-acrylamido-2-methylpropanesulfonic acid, wherein the sulfonic acid group may be present wholly or in part as a sodium, potassium, ammonium, mono- or triethanolammonium salt.

The homopolymer of 2-acrylamido-2-methylpropanesulfonic acid is more preferred, and is commercially obtainable for example under the name Rheothik® 11-80.

Within this embodiment it may be preferable to use copolymers of at least one anionic monomer and at least one nonionogenic monomer. With regard to anionic monomers, reference is made to the above-listed substances. Preferred nonionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and in particular polyacrylamide copolymers with monomers containing sulfonic acid groups. A particularly preferred anionic copolymer consists of 70 to 55 mol % acrylamide and 30 to 45 mol % 2-acrylamido-2-methylpropanesulfonic acid, wherein the sulfonic acid group is present wholly or in part as a sodium, potassium, ammonium, mono- or triethanolammonium salt. This copolymer may also be present in crosslinked form, wherein polyolefinically unsaturated compounds such as tetraallyloxyethane, allyl sucrose, allyl pentaerythritol and methylene bisacrylamide are preferably used as the crosslinking agents. Such a polymer is present in the commercial product Sepigel®305 from SEPPIC. Use of this compound, which contains in addition to the polymer component a hydrocarbon mixture (C₁₃-C₁₄ isoparaffin) and a nonionogenic emulsifier (Laureth-7), has proven particularly advantageous for the purposes of the teaching according to the invention.

The sodium acryloyldimethyl taurate copolymers distributed under the name Simulgel® 600 as a compound with isohexadecane and polysorbate-80 have also proven particularly effective according to the invention.

Anionic homopolymers which are likewise preferred are uncrosslinked and crosslinked polyacrylic acids. In this case, allyl ethers of pentaerythritol, of sucrose and of propylene may be preferred crosslinking agents. Such compounds are commercially obtainable for example under the trademark Carbopol®.

Copolymers of maleic anhydride and methyl vinyl ether, in particular those comprising crosslinks, are also color-preserving polymers. A maleic acid-methyl vinyl ether copolymer crosslinked with 1,9-decadiene is commercially obtainable under the name Stabileze® QM.

In cosmetic compositions which are preferred according to the invention, the anionic polymer is selected from:
polymers which comprise carboxyl units which are derived from mono- or dicarboxylic acid monomers of the following formula:

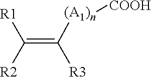

in which n means 0 or an integer from 1 to 10, A₁ is a methylene group which is optionally attached via a heteroatom, such as oxygen or sulfur, to the carbon atom of the unsaturated group or, if n is greater than 1, to the adjacent methylene group, R¹ means a hydrogen atom, phenyl or benzyl, R² means a hydrogen atom, a lower alkyl group or carboxy and R³ means a hydrogen atom, a lower alkyl group, the group —CH₂COOH, phenyl or benzyl;
polymers which comprise units which are derived from a sulfonic acid, such as vinylsulfonic acid, styrenesulfonic acid, acrylamidoalkylsulfonic acid.

In cosmetic compositions which are more preferred according to the invention, the anionic polymer is selected from:
copolymers of acrylic acid;
copolymers derived from crotonic acid;
polymers which are derived from maleic acid or maleic anhydride, fumaric acid or itaconic acid and vinyl esters, vinyl ethers, vinyl halides, phenylvinyl derivatives, acrylic acid and the esters thereof;
copolymers of methacrylic acid and methyl methacrylate;
the copolymer of methacrylic acid and ethyl acrylate;
vinyl acetate/crotonic acid copolymer;
vinyl acetate/crotonic acid/polyethylene glycol terpolymer.

Amphoteric polymers may furthermore be used as a polymer component for increasing the action of the active substance combination according to the invention. The term amphoteric polymers includes not only those polymers which contain in each molecule both free amino groups and free —COOH or —SO₃H groups and are capable of forming internal salts, but also zwitterionic polymers, which contain in each molecule quaternary ammonium groups and —COO⁻ or —SO₃⁻ groups, and those polymers which contain —COOH or —SO₃H groups and quaternary ammonium groups.

One example of an amphoteric polymer usable according to the invention is the acrylic resin obtainable under the name Amphomer®, which is a copolymer of tert.-butylaminoethyl methacrylate, N-(1,1,3,3-tetramethylbutyl)acrylamide and two or more monomers from the group acrylic acid, methacrylic acid and the simple esters thereof.

Further amphoteric polymers usable according to the invention are the compounds stated in British published patent application 2 104 091, European published patent application 47 714, European published patent application 217 274, European published patent application 283 817 and German published patent application 28 17 369.

Amphoteric polymers which are preferably used are those polymers which are substantially composed of
(a) monomers with quaternary ammonium groups of general formula (IV),

R²²—CH═CR²³—CO—Z—(CₙH₂ₙ)—N⁽⁺⁾R²⁴R²⁵R²⁶A⁽⁻⁾ (IV)
in which R²² and R²³ mutually independently denote hydrogen or a methyl group and R²⁴, R²⁵ and R²⁶ mutually independently denote alkyl groups having 1 to 4 carbon atoms, Z is an NH group or an oxygen atom, n is an integer from 2 to 5 and A⁽⁻⁾ is the anion of an organic or inorganic acid and (b) monomeric carboxylic acids of general formula (V),

R²⁷—CH═CR²⁸—COOH (V) in which R²⁷ and R²⁸ are mutually independently hydrogen or methyl groups.

These compounds may be used according to the invention both directly and in salt form, which is obtained by neutralizing the polymers, for example with an alkali metal hydroxide. Particularly preferred polymers are those in which monomers of type (a) are used, in which R²⁴, R²⁵ and R²⁶ are methyl groups, Z is an NH group and A⁽⁻⁾ is a halide, or ethoxysulfate ion; acrylamidopropyltrimethylammonium chloride is a more preferred monomer (a). Acrylic acid is preferably used as monomer (b) for the stated polymers.

In cosmetic compositions which are more preferred according to the invention, the amphoteric polymer is selected from the polymers which comprise units derived from:
a) at least one monomer which is selected from acrylamides or methacrylamides which are substituted with an alkyl group on the nitrogen;
b) at least one acid comonomer which comprises one or more reactive carboxy groups; and
c) at least one basic comonomer, such as esters of acrylic acid and methacrylic acid with primary, secondary, tertiary and quaternary amino substituents, and the quaternization product of dimethylaminoethyl methacrylate with dimethyl sulfate or diethyl sulfate.

In a third variant, the agents according to the invention may furthermore contain nonionogenic polymers.

Suitable nonionogenic polymers are for example:

vinylpyrrolidone/vinyl ester copolymers, as are distributed for example under the tradename Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methylhydroxypropylcellulose, as are distributed for example under the tradename Culminal® and Benecel® (AQUALON).

shellac

polyvinylpyrrolidones, as are distributed for example under the tradename Luviskol® (BASF).

siloxanes. These siloxanes may be either water-soluble or water-insoluble. Both volatile and non-volatile siloxanes are suitable, wherein non-volatile siloxanes are taken to be those compounds whose boiling point at standard pressure is above 200° C. Preferred siloxanes are polydialkylsiloxanes, such as for example polydimethylsiloxane, polyalkylarylsiloxanes, such as for example polyphenylmethylsiloxane, ethoxylated polydialkylsiloxanes and polydialkylsiloxanes, which contain amine and/or hydroxy groups.

In cosmetic compositions which are more preferred according to the invention, the nonionic polymer is selected from:
polyalkyloxazolines;

homopolymers of vinyl acetate;

copolymers of vinyl acetate and an acrylic ester;

copolymers of vinyl acetate and ethylene;

copolymers of vinyl acetate and a maleic acid ester;

copolymers of polyethylene and maleic anhydride;

homopolymers of alkyl acrylates and homopolymers of alkyl methacrylates;

copolymers of acrylic esters;

copolymers of acrylonitrile and a nonionic monomer; and

copolymers of alkyl acrylate and urethane.

Cationic polymers should be taken to mean polymers which comprise groups in the main and/or side chain which may be “temporarily” or “permanently” cationic. Polymers which are designated “permanently cationic” according to the invention are those which, irrespective of the pH value of the agent, comprise a cationic group. As a rule, these are polymers which contain a quaternary nitrogen atom, for example in the form of an ammonium group. Preferred cationic groups are quaternary ammonium groups. Polymers which have proven particularly suitable are in particular those in which the quaternary ammonium group is attached via a C₁₋₄ hydrocarbon group to a main polymer chain synthesized from acrylic acid, methacrylic acid or the derivatives thereof.

Homopolymers of general formula (III),

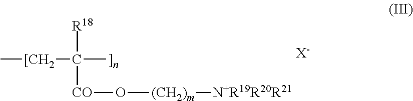

in which R¹⁸ is —H or —CH₃, R¹⁹, R²⁰ and R²¹ are mutually independently selected from C₁₋₄ alkyl, alkenyl or hydroxyalkyl groups, m=1, 2, 3 or 4, n is a natural number and X⁻ a physiologically acceptable organic or inorganic anion, and copolymers substantially consisting of the monomer units listed in formula (III) and nonionogenic monomer units are more preferred cationic polymers. In the context of these polymers, those which are preferred according to the invention are those for which at least one of the following conditions applies:

R¹⁸ denotes a methyl group

R¹⁹, R²⁰ and R²¹ denote methyl groups

m has the value 2.

Physiologically acceptable counterions X⁻ which may, for example, be considered are halide ions, sulfate ions, phosphate ions, methosulfate ions and organic ions such as lactate, citrate, tartrate and acetate ions. Halide ions, in particular chloride, are preferred.

A particularly suitable homopolymer is poly(methacryloyloxyethyl-trimethylammonium chloride), which may if desired be crosslinked, with the INCI name of Polyquaternium-37. Crosslinking may if desired proceed with the assistance of olefinically polyunsaturated compounds, for example divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylenebisacrylamide is a preferred crosslinking agent.

The homopolymer is preferably used in the form of a nonaqueous polymer dispersion which should have a polymer fraction of no less than 30 wt. %. Such polymer dispersions are commercially available under the names Salcare® SC 95 (approx. 50% polymer fraction, further components: mineral oil (INCI name: Mineral Oil) and tridecyl-polyoxypropylene-polyoxyethylene ether (INCI name: PPG-1-Trideceth-6)) and Salcare® SC 96 (approx. 50% polymer fraction, further components: mixture of diesters of propylene glycol with a mixture of caprylic and capric acid (INCI name: Propylene Glycol Dicaprylate/Dicaprate) and tridecyl-polyoxypropylene-polyoxyethylene ether (INCI name: PPG-1-Trideceth-6)).

Copolymers with monomer units according to formula (III) preferably contain acrylamide, methacrylamide, acrylic acid C₁₋₄ alkyl esters and methacrylic acid C₁₋₄ alkyl esters as nonionogenic monomer units. Acrylamide is more preferred among these nonionogenic monomers. These copolymers, as described above for the homopolymers, may also be crosslinked. A copolymer which is preferred according to the invention is crosslinked acrylamide-methacryloyloxyethyltrimethylammonium chloride copolymer. Such copolymers, in which the monomers are present in a weight ratio of approx. 20:80, are commercially available as approx. 50% nonaqueous polymer dispersions under the name Salcare® SC 92.

Further preferred cationic polymers are for example
quaternized cellulose derivatives, as are commercially available under the names Celquat® and Polymer JR®. The compounds Celquat® H 100, Celquat® L 200 and Polymer JR®400 are preferred quaternized cellulose derivatives,
cationized honey, for example the commercial product Honeyquat® 50,
cationic guar derivatives, such as in particular the products distributed under the trade names Cosmedia® Guar and Jaguar®,
polysiloxanes with quaternary groups, such as for example the commercially obtainable products Q2-7224 (manufacturer: Dow Corning; a stabilized trimethylsilylamodimethicone), Dow Corning® 929 Emulsion (containing a hydroxylamino-modified silicone which is also designated an amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxane, Quaternium-80).
polymeric dimethyldiallylammonium salts and the copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as for example vinylpyrrolidone-dimethylaminoethyl methacrylate copolymers quaternized with diethyl sulfate. Such compounds are commercially available under the names Gafquat®734 and Gafquat®755,
vinylpyrrolidone-vinylimidazolium methochloride copolymers, as are offered for sale under the names Luviquat® FC 370, FC 550, FC 905 and HM 552,
quaternized polyvinyl alcohol,
   together with polymers known under the names
Polyquaternium-2,
Polyquatemium-17,
Polyquaternium-18 and
Polyquaternium-27 with quaternary nitrogen atoms in the polymer main chain.

The polymers known under the names Polyquatemium-24 (commercial product, for example Quatrisoft® LM 200) may also be used as cationic polymers. Copolymers of vinylpyrrolidone, as are available as commercial products Copolymer 845 (manufacturer: ISP), Gaffix® VC 713 (manufacturer: ISP), Gafquat®ASCP 1011, Gafquat®HS 110, Luviquat® 8155 and Luviquat® MS 370 may likewise be used according to the invention. Further cationic polymers according to the invention are “temporarily cationic” polymers. These polymers conventionally contain an amino group which at specific pH values assumes the form of a quaternary ammonium group and is thus cationic. Chitosan and the derivatives thereof, as are readily commercially available for example under the trade names Hydagen® CMF, Hydagen® HCMF, Kytamer® PC and Chitolam® NB/101, are for example preferred. Chitosans are deacetylated chitins which are commercially available in various degrees of deacetylation and various degrees of degradation (molecular weights). The production thereof is described, for example, in DE 44 40 625 A1 and in DE 1 95 03 465 A1 Particularly highly suitable chitosans exhibit a degree of deacetylation of at least 80% and a molecular weight of 5·10⁵ to 5·10⁶ (g/mol).

In order to produce preparations according to the invention, the chitosan can be converted into the salt form. This may proceed by dissolution in dilute aqueous acids. Suitable acids are both mineral acids such as for example hydrochloric acid, sulfuric acid and phosphoric acid and organic acids, for example low molecular weight carboxylic acids, polycarboxylic acids and hydroxycarboxylic acids. Relatively high molecular weight alkylsulfonic acids or alkylsulfuric acids or organophosphoric acids may furthermore be used, provided that they have the necessary physiological acceptability. Suitable acids for converting the chitosan into the salt farm are for example acetic acid, glycolic acid, tartaric acid, malic acid, citric acid, lactic acid, 2-pyrrolidinone-5-carboxylic acid, benzoic acid or salicylic acid. Low molecular weight hydroxycarboxylic acids such as for example glycolic acid or lactic acid are preferably used.

To summarize, preferred cosmetic compositions according to the invention are characterized in that the cationic amphiphilic polymers are selected from quaternized cellulose derivatives and polyacrylates with aminated side chains.

Further preferred cosmetic compositions are characterized in that the cationic polymers are selected from those polymers which have units with primary, secondary, tertiary and/or quaternary amino groups, which units are either part of the polymer main chain or are on lateral substituents attached directly thereto.

Still more preferred cosmetic compositions are characterized in that the cationic polymer is selected from cationic cyclopolymers, cationic polysaccharides, quaternary polymers of vinylpyrrolidone and vinylimidazole and mixtures thereof.

Likewise preferred cosmetic compositions are characterized in that the cyclopolymer is selected from homopolymers of diallyldimethylammonium chloride and the copolymers of diallyldimethylammonium chloride and acrylamide.

Further preferred cosmetic compositions are characterized in that the cationic polysaccharides are selected from hydroxyethylcelluloses which have reacted with an epoxide substituted with a trimethylammonium group.

Still more preferred cosmetic compositions are characterized in that the cationic polysaccharides are selected from guar gums modified with a 2,3-epoxypropyltrimethylammonium salt.

It is also possible according to the invention for the preparations used to contain a plurality of, in particular two different, identically charged polymers and/or in each case one ionic and one amphoteric and/or nonionic polymer.

The term polymer should be taken according to the invention likewise to mean specific preparations of polymers such as spherical polymer powders. Various methods are known for producing such microspheres from various monomers, for example by special polymerization methods or by dissolving the polymers in a solvent and spraying into a medium in which the solvent can evaporate or diffuse out of the particles. Such a method is known, for example, from EP 466 986 B1. Suitable polymers are for example polycarbonates, polyurethanes, polyacrylates, polyolefins, polyesters or polyamides. Particularly suitable such spherical polymer powders are those having a primary particle diameter of less than 1 vim. Such products based on a polymethacrylate copolymer are, for example, commercially available under the trademark Polytrap®Q5-6603 (Dow Corning). Other polymer powders, for example based on polyamides (Nylon 6, Nylon 12), are obtainable with a particle size of 2-10 μm (90%) and a specific surface area of approx. 10 m²/g under the trade name Orgasol® 2002 DU Nat Cos(Atochem S.A., Paris). Further spherical polymer powders which are suitable for the purpose according to the invention are, for example, polymethacrylates (Micropearl M) from SEPPIC or (Plastic Powder A) from NIKKOL, styrene-divinylbenzene copolymers (Plastic Powder FP) from NIKKOL, polyethylene and polypropylene powders (ACCUREL EP 400) from AKZO or also silicone polymers (Silicone Powder X2-1605) from Dow Corning or also spherical cellulose powders.

The polymers are present in the agents according to the invention preferably in quantities of from 0.01 to 10 wt. %, relative to the total agent. Quantities of 0.1 to 5, in particular of 0.1 to 3 wt. %, are more preferred.

Trade names of silicones preferably used as conditioning agents are for example
Versicol E or K (Ciba)
Ultrahold (BASF)
Reten 421, 423 or 425 (Hercules)
Quadramer (American Cyanamid)
Acrylidone LM (ISP)
Luvimer 100 P (BASF)
28-29-30, 26-13-14 and 28-13-10 (National Starch)
Gantrez AN or ES (ISP)
Flexan 500 and Flexan 130 (National Starch)
Cosmedia Polymer HSP 1180 (Cognis)
Ultrahold Strong (BASF)
Resin 28-29-30 (National Starch)
Gantrez ES 425 (ISP)
Eudragit L (Rohm Pharma)
Luvimer MAEX or MAE (BASF)
Luviset CA 66 (BASF)
Aristoflex A (BASF)
Acrylidone LM (ISP)
Polyquart KE 3033 (Cognis)
Merquat 280, Merquat 295 and Merquat Plus 3330 (Nalco)
Diaformer Z301 (Sandoz)
N-carboxymethylchitosan and N-carboxybutylchitosan “Evalsan” (Jan Dekker)
Peox 50 000, Peox 200 000 and Peox 500 000
Appretan EM (Hoechst)
Rhodopas A 012 (Rhodia Chimie)
Rhodopas AD 310 (Rhodia Chimie)
Appretan TV (Hoechst)
Appretan MB Extra (Hoechst)
Micropearl RQ 750 (Matsumoto) and Luhydran A 848 S (BASF)
Primal AC-261 K and Eudragit NE 30 D (Rohm & Haas)
Acronal 601, Luhydran LR 8833 and 8845 (BASF)
Appretan N 9213 or N 9212 (Hoechst)
Nipol LX 531 B (Nippon Zeon)
CJ 0601 B (Rohm & Haas)
Acrysol RM 1020 and Acrysol RM 2020 (Rohm & Haas)
Uraflex XP 401 UZ and Uraflex XP 402 UZ (DSM Resins)
8538-33 (National Starch)
Estapor LO 11 (Rhodia Chimie)
Vidogum GH 175 (Unipectine)
Jaguar C (Meyhall)
Jaguar HP8, Jaguar HP60 and Jaguar HP120, Jaguar DC 293 and Jaguar HP 105 (Meyhall)
Galactasol 4H₄FD2 (Aqualon)
Hercofloc (Hercules)
Bina Quat P 100 (Ciba)
“Gafquat” (ISP), for example “Gafquat® 734” or “Gafquat® 755”
Copolymer 845, 958 and 937 (ISP)
Gaffix®VC713(ISP)
Styleze®CC 10 (ISP)
Gafquat® HS 100 (ISP)
(JR 400, JR 125, JR 30M) or (LR 400, LR 30M) (Amerchol)
Celquat® L 200 and Celquat® H 100 (National Starch)
Jaguar® C13S, Jaguar® C15, Jaguar® C17 and Jaguar® C162 (Meyhall)
Cartaretine® F, F4 or F8 (Sandoz)
PD 170 or Delsette® 101 (Hercules)
Mirapol® A 15, Mirapol® AD1, Mirapol® AZ1 and Mirapol® 175 (Miranol)
Luviquat® FC 905, FC 550 and FC 370 (BASF)
Polyquart® H (Cognis)
Salcare® SC 92 (Ciba)
Salcare® SC 95 and Salcare® SC 96 (Ciba)
Merquat® 100, Merquat® 550 and Merquat® S (Nalco)
Quat-Pro E (Maybrook)
Quat-Pro S (Maybrook)
Crotein® BTA, Croquat® L, Croquat® M, Croquat® S, Crotein® Q (Croda)
Lexein® QX 3000 (Inolex)
Hydrotriticum WQ or QM, Hydrotriticum QL, Hydrotriticum QS.

Irrespective of the conditioning agent(s) selected, preferred cosmetic compositions according to the invention are those which contain the conditioning agent(s) in a total quantity of 0.001 to 20 wt. %, preferably of 0.01 to 15 wt. %, further preferably of 0.1 to 10 wt. % and in particular of 0.25 to 5 wt. %, relative to the total weight of the composition.

Depending on the intended application, the agents according to the invention contain further essential ingredients. Cleaning or conditioning compositions such as for example shampoos or conditioners contain at least one surfactant, wherein, depending on the area of application, surface-active substances are known as surfactants or as emulsifiers and are selected from anionic, cationic, zwitterionic, ampholytic and nonionic surfactants and emulsifiers.

Cosmetic agents which are preferred according to the invention are characterized in that, relative to the weight thereof, they contain 0.5 to 70 wt %, preferably 1 to 60 wt % and in particular 5 to 25 wt. % of anionic and/or nonionic and/or cationic and/or amphoteric surfactant(s).

Anionic surfactants and emulsifiers which are suitable in compositions according to the invention are any anionic surface-active substances suitable for use on the human body. These are characterized by an anionic water-solubilizing group such as for example a carboxylate, sulfate, sulfonate or phosphate group and a lipophilic alkyl group having approx. 8 to 30 C atoms. The molecule may additionally contain glycol or polyglycol ether groups, ester, ether and amide groups and hydroxyl groups. Examples of suitable anionic surfactants and emulsifiers are, in each case in the form of sodium, potassium and ammonium and the mono-, di- and trialkanolammonium salts having 2 to 4 C atoms in the alkanol group,
linear and branched fatty acids with 8 to 30 C atoms (soaps),
ether carboxylic acids of formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides with 8 to 24 C atoms in the acyl group,
acyl isethionates with 8 to 24 C atoms in the acyl group,
linear alkane sulfonates with 8 to 24 C atoms,
linear alpha-olefin sulfonates with 8 to 24 C atoms,
alpha-sulfofatty acid methyl esters of fatty acids with 8 to 30 C atoms,
acyl glutamates of formula (I),

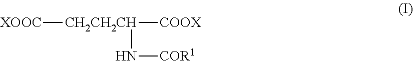

in which R¹CO denotes a linear or branched acyl residue with 6 to 22 carbon atoms and 0, 1, 2 or 3 double bonds and X denotes hydrogen, an alkali metal and/or alkaline earth metal, ammonium, alkylammonium, alkanolammonium or glucammonium, for example acyl glutamates which are derived from fatty acids with 6 to 22, preferably 12 to 18 carbon atoms, such as for example C_{12/14} or C_{12/18} coconut fatty acid, lauric acid, myristic acid, palmitic acid and/or stearic acid, in particular sodium N-cocoyl L-glutamate and sodium N-stearoyl L-glutamate, esters of a hydroxy-substituted di- or tricarboxylic acid of general formula (II),

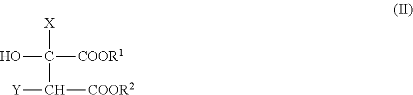

in which X═H or a —CH₂COOR group, Y═H or —OH under the condition that Y═H, if X═—CH₂COOR, R, R¹ and R² mutually independently mean a hydrogen atom, an alkali or alkaline earth metal cation, an ammonium group, the cation of an organoammonium base or a residue Z which originates from a polyhydroxylated organic compound which is selected from the group of etherified (C₆-C₁₈) alkyl polysaccharides with 1 to 6 monomeric saccharide units and/or of etherified aliphatic (C₆-C₁₆) hydroxyalkyl polyols with 2 to 16 hydroxyl residues, with the proviso that at least one of the groups R, R¹ or R² is a residue Z,
esters of sulfosuccinic acid or sulfosuccinates of general formula (III),

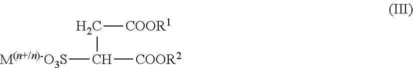

in which M^{(n+/n)} represents, where n=1, a hydrogen atom, an alkali metal cation, an ammonium group or the cation of an organoammonium base and, where n=2, an alkaline earth metal cation and R¹ and R² mutually independently mean a hydrogen atom, an alkali or alkaline earth metal cation, an ammonium group, the cation of an organoammonium base or a residue Z which originates from a polyhydroxylated organic compound which is selected from the group of etherified (C₆-C₁₈) alkyl polysaccharides with 1 to 6 monomeric saccharide units and/or of etherified aliphatic (C₆-C₁₆) hydroxyalkyl polyols with 2 to 16 hydroxyl residues, with the proviso that at least one of the groups R¹ or R² is a residue Z,
sulfosuccinic acid mono- and dialkyl esters with 8 to 24 C atoms in the alkyl group and sulfosuccinic acid monoalkyl polyoxyethyl esters with 8 to 24 C atoms in the alkyl group and 1 to 6 oxyethyl groups,
alkyl sulfates and alkyl polyglycol ether sulfates of formula R—O(CH₂—CH₂O)ₓ—OSO₃H, in which R is a preferably linear alkyl group with 8 to 30 C atoms and x=0 or 1 to 12,

mixed surface-active hydroxysulfonates according to DE-A-37 25 030,
esters of tartaric acid and citric acid with alcohols which are addition products of approx. 2-15 molecules of ethylene oxide and/or propylene oxide onto C₈₋₂₂ fatty alcohols,
alkyl and/or alkenyl ether phosphates,
sulfated fatty acid alkylene glycol esters,
monoglyceride sulfates and monoglyceride ether sulfates.

Preferred anionic surfactants and emulsifiers are acyl glutamates, acyl isethionates, acyl sarcosinates and acyl taurates, in each case with a linear or branched acyl residue with 6 to 22 carbon atoms and 0, 1, 2 or 3 double bonds, which residue is selected in more preferred embodiments from an octanoyl, decanoyl, lauroyl, myristoyl, palmitoyl and stearoyl residue, esters of tartaric acid, citric acid or succinic acid or of the salts of these acids with alkylated glucose, in particular the products with the INCI names Disodium Coco-Glucoside Citrate, Sodium Coco-Glucoside Tartrate and Disodium Coco-Glucoside Sulfosuccinate, alkyl polyglycol ether sulfates and ether carboxylic acids with 8 to 18 C atoms in the alkyl group and up to 12 ethoxy groups per molecule, sulfosuccinic acid mono- and dialkyl esters with 8 to 18 C atoms in the alkyl group and sulfosuccinic acid monoalkyl polyoxyethyl esters with 8 to 18 C atoms in the alkyl group and 1 to 6 ethoxy groups.

Those surface-active compounds which bear at least one quaternary ammonium group and at least one —COO⁽⁻⁾ or —SO₃⁽⁻⁾ group on each molecule are designated as zwitterionic surfactants and emulsifiers. Particularly suitable zwitterionic surfactants and emulsifiers are “betaines” such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines with in each case 8 to 18 C atoms in the alkyl or acyl group and cocoacylaminoethylhydroxyethylcarboxymethyl glycinate. One preferred zwitterionic surfactant is the fatty acid amide derivative known by the INCI name Cocamidopropyl Betaine.

Preferred anionic surfactants are alkyl sulfates, alkyl polyglycol ether sulfates and ether carboxylic acids having 10 to 18 C atoms in the alkyl group and up to 12 glycol ether groups per molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups.

More preferred anionic surfactants are the alkali metal or ammonium salts of lauryl ether sulfate with a degree of ethoxylation of 2 to 4 EO.

More preferred cosmetic agents according to the invention are characterized in that, relative to the weight thereof, they contain 0.1 to 20 wt. %, preferably 0.25 to 17.5 wt. % and in particular 5 to 15 wt. % of anionic surfactant(s), more preferably fatty alcohol ether sulfates of formula

H₃C—(CH₂)ₙ—(OCH₂CH₂)ₖ—OSO₃⁻M⁺

in which n denotes values of 5 to 21, preferably of 7 to 19, more preferably of 9 to 17 and in particular of 11 to 13 and k denotes values of 1, 2, 3, 4, 5, 6, 7, 8, 9 or 10, preferably 1, 2 or 3 and in particular 2, and M denotes a cation from the group Na⁺, K⁺, NH₄⁺, ½Mg²⁺, ½Zn²⁺, preferably Na⁺.

Those surface-active compounds which bear at least one quaternary ammonium group and at least one —COO⁽⁻⁾ or —SO₃⁽⁻⁾ group on each molecule are designated as zwitterionic surfactants and emulsifiers. Particularly suitable zwitterionic surfactants and emulsifiers are “betaines” such as N-alkyl-N,N-dimethylammonium glycinates, for example co coalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines with in each case 8 to 18 C atoms in the alkyl or acyl group and cocoacylaminoethylhydroxyethylcarboxymethyl glycinate. One preferred zwitterionic surfactant is the fatty acid amide derivative known by the INCI name Cocamidopropyl Betaine.

Ampholytic surfactants and emulsifiers are taken to mean those surface-active compounds which, in addition to a C₈-C₂₄ alkyl or acyl group, contain at least one free amino group and at least one —COOH or —SO₃H group and are capable of forming internal salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarco sines, 2-alkylaminopropionic acids and alkylaminoacetic acids with in each case approx. 8 to 24 C atoms in the alkyl group. Particularly preferred ampholytic surfactants are N-cocoalkyl aminopropionate, cocoacylaminoethyl aminopropionate and C₁₂₋₁₈ acyl sarcosine.

More preferred cosmetic agents according to the invention are characterized in that they contain amphoteric surfactant(s) from the groups of N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids having in each case approx. 8 to 24 C atoms in the alkyl group, alkylaminoacetic acids having in each case approx. 8 to 24 C atoms in the alkyl group, N-cocoalkyl aminopropionate, cocoacylaminoethyl aminopropionate C12-C₁₋₈ acyl sarcosine, N-alkyl-N,N-dimethylammonium glycinates, for example cocoalkyldimethyl-ammonium glycinate, N-acylaminopropyl-N,N-dimethylammonium glycinates, for example cocoalkylaminopropyldimethylammonium glycinate, 2-alkyl-3-carboxymethyl-3-hydroxyethylimidazolines having in each case 8 to 18 C atoms in the alkyl or acyl group, cocoacylaminoethylhydroxyethylcarboxymethyl glycinate, the compounds known by the INCI name Cocamidopropyl Betaine, the compounds known by the INCI name Disodium Cocoamphodiacetates, wherein preferred agents contain the amphoteric surfactant(s) in quantities of 0.5 to 9 wt. %, preferably of 0.75 to 8 wt. % and in particular of 1 to 7.5 wt. %, in each case relative to the entire agent.

More preferred cosmetic agents contain betaines of formula (Bet-I)

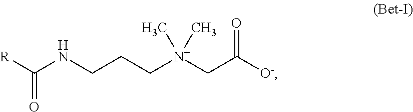

in which R denotes a linear or branched, saturated or mono- or polyunsaturated alkyl or alkenyl residue with 8 to 24 carbon atoms as amphoteric surfactants.

According to INCI nomenclature, these surfactants are designated Amidopropylbetaines, wherein those representatives which are derived from coconut fatty acids are preferred and are designated Cocamidopropylbetaines. Surfactants of formula (Bet-I) which are a mixture of the following representatives are more preferably used according to the invention: H₃C—(CH₂)₇—C(O)—NH—(CH₂)₃N⁺(CH₃)₂CH₂COO, H₃C—(CH₂)₉—C (O)—NH—(CH₂)₃N⁺(CH₃)₂CH₂COO⁻, H₃C—(CH₂)₁₁—C(O)—NH—(CH₂)₃N⁺(CH₃)₂CH₂COO⁻, H₃C—(CH₂)₁₃—C(O)—NH—(CH₂)₃N⁺(CH₃)₂CH₂COO⁻, H₃C—(CH₂)₁₅—C(O)—NH—(CH₂)₃N⁺(CH₃)₂CH₂COO⁻, H₃C—(CH₂)₇—CH═CH—(CH₂)₇—C(O)—NH—(CH₂)₃N⁺(CH₃)₂CH₂COO⁻.

Surfactants of formula (Bet-I) are more preferably used within relatively narrow quantity ranges. Preferred agents according to the invention are here those which, relative to the weight thereof, contain 0.25 to 8 wt. %, further preferably 0.5 to 7 wt. %, further preferably 0.75 to 6.5 wt. % and in particular 1 to 5.5 wt. % of surfactant(s) of formula (Bet-I).

In addition to or instead of the amphoteric surfactant(s) of formula (Bet-I), the cosmetic agents according to the invention may particularly preferentially contain betaines of formula (Bet-II)

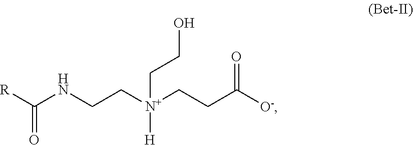

in which R denotes a linear or branched, saturated or mono- or polyunsaturated alkyl or alkylene residue with 8 to 24 carbon atoms as amphoteric surfactants.

According to INCI nomenclature, these surfactants are designated Amphoacetates, wherein those representatives which are derived from coconut fatty acids are preferred and are designated Cocoamphoacetates.

For manufacturing reasons, surfactants of this type always also contain betaines of formula (Bet-IIa)

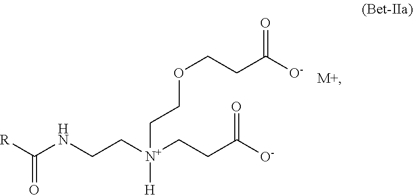

in which R denotes a straight-chain or branched, saturated or mono- or polyunsaturated alkyl or alkenyl residue with 8 to 24 carbon atoms and M denotes a cation.

According to INCI nomenclature, these surfactants are designated Amphodiacetates, wherein those representatives which are derived from coconut fatty acids are preferred and are designated Cocoamphodiacetates.

Surfactants of formula (Bet-II) which are more preferably used according to the invention are those which are a mixture of the following representatives:
H₃C—(CH₂)₇—C(O)—NH—(CH₂)₂NH⁺(CH₂CH₂₀H)CH₂CH₂COO⁻
H₃C—(CH₂)₉—C(O)—NH—(CH₂)₂NH⁺(CH₂CH₂₀H)CH₂CH₂COO⁻
H₃C—(CH₂)₁₁—C(O)—NH—(CH₂)₂NH⁺(CH₂CH₂₀H)CH₂CH₂COO⁻
H₃C—(CH₂)₁₃—C(O)—NH—(CH₂)₂NH⁺(CH₂CH₂₀H)CH₂CH₂COO⁻
H₃C—(CH₂)₁₅—C(O)—NH—(CH₂)₂NH⁺(CH₂CH₂OH)CH₂CH₂COO⁻
H₃C—(CH₂)₇—CH═CH—(CH₂)₇—C(O)—NH—(CH₂)₂NH⁺(CH₂CH₂OH)CH₂CH₂COO⁻.

Surfactants of formula (Bet-II) are more preferably used within relatively narrow quantity ranges. Preferred agents according to the invention are here those which, relative to the weight thereof, contain 0.25 to 8 wt. %, further preferably 0.5 to 7 wt. %, further preferably 0.75 to 6.5 wt. % and in particular 1 to 5.5 wt. % of surfactant(s) of formula (Bet-II).

To summarize, preferred cosmetic agents according to the invention are those in which the residue R in the formulae (Bet-I) and (Bet-II) is selected from H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇—, or mixtures of these.

More preferred nonionic surfactants are alkyl polyglycosides. Preferred cosmetic agents according to the invention are therefore those which, relative to the weight thereof, contain as nonionic surfactants 0.1 to 20 wt. % of alkyl polyglycosides of general formula RO—(Z)ₓ, wherein R denotes alkyl, Z denotes sugar and x denotes the number of sugar units.

Alkyl polyglycosides (APG) are nonionic surfactants which are entirely produced from renewable raw materials (sugar building blocks, predominantly glucose, for example from corn starch, and fatty alcohol, for example from coconut oil). Alkyl polyglycosides are obtainable by an acidically catalyzed reaction (Fischer reaction) of sugars, in particular glucose (or starch) or of butyl glycosides with fatty alcohols.

Complex mixtures of alkyl monoglucoside (alkyl α-D- and β-D-glucopyranoside and small proportions of glucofuranoside), alkyl diglucosides (alkyl isomaltosides, alkyl maltosides etc.) and alkyl oligoglucosides (alkyl maltotriosides, alkyl tetraosides etc.) are obtained as a result. The average degree of polymerization of commercial products, the alkyl residues of which are in the C₈-C₁₆ range, amounts to 1.2-1.5.

Alkyl polyglycosides which are preferred according to the invention are those which correspond to general formula RO—(Z)ₓ, wherein R denotes alkyl, Z denotes sugar and x denotes the number of sugar units. More preferred such alkyl polyglycosides are those in which R

substantially consists of C₈ and C₁₀ alkyl groups,

substantially consists of C₁₂ and C₁₄ alkyl groups,

substantially consists of C₈ to C₁₆ alkyl groups or

substantially consists of C₁₂ to C₁₆ alkyl groups or

substantially consists of C₁₆ to C₁₈ alkyl groups.

Any desired mono- or oligosaccharides may be used as the sugar building block Z. Sugars with 5 or 6 carbon atoms and the corresponding oligosaccharides are conventionally used. Such sugars are for example glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is more preferred.

Alkyl polyglycosides usable according to the invention contain on average 1.1 to 5 sugar units. Alkyl polyglycosides with x values of 1.1 to 2.0 are preferred. Alkyl glycosides in which x is 1.1 to 1.8 are particularly preferred.

Further surfactants which, in particular mixed with alkyl polyglycosides, may more advantageously be used in the agents according to the invention are glutamates, aspartates and sulfoacetates. Preferred cosmetic agents according to the invention are here those which, relative to the weight thereof, contain 0.1 to 20 wt. % of fatty acid glutamates (acyl glutamates) and/or fatty acid aspartates (acyl aspartates) and/or alkyl sulfoacetates (sulfoacetic acid alkyl esters).

Typical examples of suitable acyl glutamates are anionic surfactants which are derived from fatty acids with 6 to 22, preferably 12 to 18 carbon atoms, such as for example C_{12/14} or C_{12/18} coconut fatty acid, lauric acid, myristic acid, palmitic acid and/or stearic acid. Sodium N-cocoyl L-glutamate and sodium N-stearoyl L-glutamate are more preferred.

The agents according to the invention may contain the alkyl and/or alkenyl oligoglucosides and the acyl glutamates in a weight ratio of 1:99 to 99:1, preferably 10:90 to 90:10 and in particular 80:20 to 50:50.

Typical examples of suitable acyl aspartates are anionic surfactants which are derived from fatty acids with 6 to 22, preferably 12 to 18 carbon atoms, such as for example C_{12/14} or C_{12/18} coconut fatty acid, lauric acid, myristic acid, palmitic acid and/or stearic acid. Sodium N-cocoyl L-aspartate and sodium N-stearoyl L-aspartate are more preferred.

The agents according to the invention may contain the alkyl and/or alkenyl oligoglucosides and the acyl aspartates likewise in a weight ratio of 1:99 to 99:1, preferably 10:90 to 90:10 and in particular 80:20 to 50:50.

It is more preferred to use the sulfoacetic acid sodium salt with the INCI name: Sodium Lauryl Sulfoacetate:

H₃C—(CH₂)₁₁—O—CO—CH₂—SO₂—ONa

Sodium lauryl sulfoacetate is a white, free-flowing powder, which gives a neutral reaction and has good foaming capacity, wetting capacity and dispersing power.

Cationic surfactants of the type including quaternary ammonium compounds, esterquats and amidoamines may be used according to the invention. Preferred quaternary ammonium compounds are ammonium halides, in particular chlorides and bromides, such as alkyltrimethylammonium chlorides, dialkyldimethylammonium chlorides and trialkylmethylammonium chlorides. The long alkyl chains of these surfactants preferably have 10 to 18 carbon atoms, as for example in cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further preferred cationic surfactants are the imidazolium compounds known under the INCI names Quatemium-27 and Quaternium-83.

More preferred hair cleaning and conditioning agents according to the invention are characterized in that, relative to the weight thereof, they contain as cationic conditioning substance 0.05 to 7.5 wt. %, preferably 0.1 to 5 wt. %, more preferably 0.2 to 3.5 wt. % and in particular 0.25 to 2.5 wt. % of cationic surfactant(s) from the group of quaternary ammonium compounds and/or esterquats and/or amidoamines, wherein preferred cationic surfactant(s) is/are selected from alkyltrimethylammonium chlorides with preferably 10 to 18 carbon atoms in the alkyl residue and/or dialkyldimethylammonium chlorides with preferably 10 to 18 carbon atoms in the alkyl residue and/or trialkylmethylammonium chlorides with preferably 10 to 18 carbon atoms in the alkyl residue and/or cetyltrimethylammonium chloride and/or stearyltrimethylammonium chloride and/or distearyldimethylammonium chloride and/or lauryldimethylammonium chloride and/or lauryldimethylbenzylammonium chloride and/or tricetylmethylammonium chloride and/or Quatemium-27 and/or Quatemium-83 and/or N-methyl-N(2-hydroxyethyl)-N,N-(ditallowacyloxyethyl)ammonium methosulfate and/or N-methyl-N(2-hydroxyethyl)-N,N-(distearoyloxyethyl)ammonium methosulfate and/or N,N-dimethyl-N,N-distearoyloxyethylammonium chloride and/or N,N-di-(2-hydroxyethyl)-N,N-(fatty acid ester ethyl)ammonium chloride.

The conditioning effects of the agents according to the invention may be still further enhanced by using specific conditioning substances. The latter are preferably selected from specific groups of per se known conditioning substances, since, with regard to formulation and conditioning effect, said conditioning substances go together outstandingly well with the silicones used according to the invention.

Cosmetic agents which are preferred according to the invention are characterized in that, relative to the weight thereof, they additionally contain conditioning substance(s) in quantities of 0.001 to 10 wt. %, preferably 0.005 to 7.5 wt. %, more preferably 0.01 to 5 wt. % and in particular 0.05 to 2.5 wt. %, wherein preferred conditioning substance(s) are selected from the group L-carnitine, and/or the salts thereof; panthenol and/or pantothenic acid; 2-furanones and/or the derivatives thereof, in particular pantolactone; taurine and/or the salts thereof; niacinamide; ubiquinone; ectoine; allantoin. In hair treatment agents according to the invention of this embodiment, the silicones are combined with at least one conditioning substance which is selected from L-camitine, and/or the salts thereof, panthenol and/or pantothenic acid, 2-furanones and/or the derivatives thereof, in particular pantolactone, taurine and/or the salts thereof, niacinamide, ubiquinones, ectoine, allantoin.

It has been found that the conditioning properties of the combination according to the invention of organopolysiloxane and conditioning agent(s) can be further increased if specific proteolipids are incorporated into the agents according to the invention. The agents according to the invention may contain at least one proteolipid of formula (P—I) as a further ingredient

R′—X—R″ (P-I),

in which
R′ denotes a straight-chain or branched, saturated or unsaturated hydrocarbon residue with 11 to 24 carbon atoms,
R″ means a protein, a peptide or a protein hydrolysate,
X denotes —C(O)O— or —N⁺(R₂^{III})R^{IV}— or —N(R^{III})R^{IV}— or —C(O)—N(R^{V})R^{VI}—,
R^{III} means —(CH₂)ₓ—CH₃ with x=0-22 and
R^{IV} means —CH₂—CH(OH)—CH₂— or —(CH₂)ₓ— with x=0-22;
R^{V} and R^{VI} mutually independently denote —H or —(CH₂)ₓ—CH₃ with x=0-22;
   with the proviso that R″ denotes keratin or a keratin hydrolysate if X denotes —C(O)O—.

The proteolipids are preferably used within specific quantities in the agents according to the invention. Preferred cosmetic agents according to the invention contain, relative to the weight thereof, 0.01 to 10 wt. %, preferably 0.02 to 5 wt. %, more preferably 0.05 to 2.5 wt. %, further preferably 0.1 to 1 wt. % and in particular 0.15 to 0.5 wt. % of proteolipid(s).

The residue R″ in formula (P-I) denotes a peptide or a protein or a protein hydrolysate.

If X=—C(O)O—, R″ is selected from the group keratin or keratin hydrolysate.

Preferred residues R″ are oligopetides which have at least one Glu-Glu-Glu amino acid sequence, wherein the amino group may assume free or protonated form and the carboxy groups may assume free or deprotonated form.

Oligopeptides for the purposes of the present application are amino acid condensation products linked by acid amide type peptide bonds and which contain at least 3 and a maximum of 25 amino acids.

In hair treatment agents of the above-described embodiment which are preferred according to the invention, the oligopeptide (=the residue R″) comprises 5 to 15 amino acids, preferably 6 to 13 amino acids, more preferably 7 to 12 amino acids and in particular 8, 9 or 10 amino acids.

The molar mass of the proteolipid present in the agents according to the invention may vary depending on whether further amino acids are attached to the Glu-Glu-Glu sequence and depending on the nature of these amino acids and as a function of the residues R′ and optionally R^{III} and R^{IV} selected.

Cosmetic agents which are preferred according to the invention are characterized in that the proteolipid has a molar mass of 1000 to 30000 Da, preferably of 1250 to 25000 Da, more preferably of 1500 to 20000 Da and in particular of 2000 to 15000 Da.

Oligopeptides which are preferably used as residue R″ are those which do not solely consist of the three glutamic acids, but instead comprise further amino acids attached to this sequence. These further amino acids are preferably selected from specific amino acids, while certain other representatives are less preferred according to the invention.

It is accordingly preferred for the residue R″ of the proteolipids used in the agents according to the invention to contain no methionine. It is further preferred for the residue R″ of the proteolipids used in the agents according to the invention to contain no cysteine and/or cystine.

It is further preferred for the residue R″ of the proteolipids used in the agents according to the invention to contain no aspartic acid and/or asparagine. It is further preferred for the residue R″ of the proteolipids used in the agents according to the invention to contain no serine and/or threonine

In contrast, it is preferred for the residue R″ of the proteolipids used in the agents according to the invention to contain tyrosine. It is further preferred for the residue R″ of the proteolipids used in the agents according to the invention to contain leucine. It is further preferred for the residue R″ of the proteolipids used in the agents according to the invention to contain isoleucine. It is further preferred for the residue R″ of the proteolipids used in the agents according to the invention to contain arginine. It is further preferred for the residue R″ of the proteolipids used in the agents according to the invention to contain valine.

Oligopeptides or amino acid sequences present in the preferred oligopeptides which are more preferred as residue R″ are described below:

A more preferred oligopeptide additionally contains tyrosine which is preferably attached via the acid function thereof to the Glu-Glu-Glu sequence. Cosmetic agents which are preferred according to the invention are therefore characterized in that the oligopeptide present as residue R″ in the proteolipids of formula (I) comprises at least one Tyr-Glu-Glu-Glu amino acid sequence, wherein the amino group may assume free or protonated folln and the carboxy groups may assume free or deprotonated form.

A further more preferred oligopeptide additionally contains isoleucine which is preferably attached via the acid function thereof to the Glu-Glu-Glu sequence Cosmetic agents which are preferred according to the invention are therefore characterized in that the oligopeptide present as residue R″ in the proteolipids of formula (I) comprises at least one Glu-Glu-Glu-Ile amino acid sequence, wherein the amino group may assume free or protonated form and the carboxy groups may assume free or deprotonated form.

Oligopeptides which comprise both of the above-stated amino acids (tyrosine and isoleucine) are preferred according to the invention. More preferred hair treatment agents according to the invention are here those in which the oligopeptide present as residue R″ in the proteolipids of formula (I) comprises at least one Tyr-Glu-Glu-Glu-Ile amino acid sequence, wherein the amino group may assume free or protonated form and the carboxy groups may assume free or deprotonated form.

Further preferred oligopeptides additionally contain arginine, which is preferably attached to isoleucine. Cosmetic agents which are preferred according to the invention are therefore characterized in that the oligopeptide present as residue R″ in the proteolipids of formula (I) comprises at least one Tyr-Glu-Glu-Glu-Ile-Arg amino acid sequence, wherein the amino groups may assume free or protonated form and the carboxy groups may assume free or deprotonated form.

Still more preferred oligopeptides additionally contain valine, which is preferably attached to arginine. Cosmetic agents which are further preferred according to the invention are therefore characterized in that the oligopeptide present as residue R″ in the proteolipids of formula (I) comprises at least one Tyr-Glu-Glu-Glu-Ile-Arg-Val amino acid sequence, wherein the amino groups may assume free or protonated form and the carboxy groups may assume free or deprotonated form.

Still more preferred oligopeptides additionally contain leucine, which is preferably attached to valine. Cosmetic agents which are further preferred according to the invention are therefore characterized in that the oligopeptide present as residue R″ in the proteolipids of formula (I) comprises at least one Tyr-Glu-Glu-Glu-Ile-Arg-Val-Leu amino acid sequence, wherein the amino groups may assume free or protonated form and the carboxy groups may assume free or deprotonated form.

More preferred oligopeptides additionally contain leucine, which is preferably attached to tyrosine. Cosmetic agents which are further preferred according to the invention are therefore characterized in that the oligopeptide present as residue R″ in the proteolipids of formula (I) comprises at least one Leu-Tyr-Glu-Glu-Glu-Ile-Arg-Val-Leu amino acid sequence, wherein the amino groups may assume free or protonated form and the carboxy groups may assume free or deprotonated form.

To summarize, cosmetic agents according to the invention which are preferred are those which contain at least one proteolipid of formula (I), in which R″ comprises at least one Leu-Tyr-Glu-Glu-Glu-Ile-Arg-Val-Leu amino acid sequence, wherein the amino groups may assume free or protonated form and the carboxy groups may assume free or deprotonated form.

As has already been mentioned, R″ is selected from the group keratin or keratin hydrolysate if X═—C(O)O—.

In all other cases, the residue R″ in formula (P-I) may denote a peptide or a protein or a protein hydrolysate, wherein protein hydrolysates are preferred. Protein hydrolysates are product mixtures which are obtained by acidically, basically or enzymatically catalyzed degradation of proteins. Protein hydrolysates of both plant and animal origin may be used according to the invention.

Animal protein hydrolysates are for example elastin, collagen, keratin, silk and milk protein hydrolysates which may also assume salt form. Such products are distributed for example under the tradenames Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

It is preferred according to the invention to use protein hydrolysates of plant origin, for example soy, almond, rice, pea, potato and wheat protein hydrolysates. Such products are obtainable, for example, under the trademarks Gluadin® (Cognis), DiaMin® (Diamalt), Lexein® (Inolex) and Crotein® (Croda).

Preferably, the residue R″ is selected from keratin or keratin hydrolysates irrespective of the X selected in formula (P-I). Preferred cosmetic agents according to the invention are characterized in that they contain at least one proteolipid of formula (P-I), in which R″ denotes keratin or a keratin hydrolysate.

In particular, preferred cosmetic agents according to the invention are those which contain at least one proteolipid of formula (P-I), in which R^{III} means —CH₃ and R^{IV} denotes —(CH₂)ₓ— with x=0, 1, 2, 3, 4, 5, 6, 7 or 8.

More preferred cosmetic agents according to the invention are further characterized in that they contain at least one proteolipid of formula (I), in which X denotes —N⁺(CH₃)₂—CH₂—CH(OH)—CH₂— and R′ denotes —(CH₂)₁₇—CH₃.

Cosmetic agents according to the invention which are likewise further preferred are characterized in that they contain at least one proteolipid of formula (P—I), in which X denotes —C(O)—O— and R′ denotes —(CH₂)₁₇—CH₃.

It has proven advantageous to use protein hydrolysates in addition to the proteolipids. Said protein hydrolysates enhance the action of the proteolipids and in turn have their effects enhanced. The protein hydrolysates have been described in detail further above as residue R″. To summarize, preferred cosmetic agents according to the invention are those which, relative to the weight thereof, additionally contain 0.01 to 10 wt. %, preferably 0.05 to 7 wt. %, more preferably 0.1 to 5 wt. %, further preferably 0.25 to 2.5 wt. % and in particular 0.5 to 2.0 wt. % of protein hydrolysate(s), preferably keratin hydrolysate(s).

For esthetic reasons, consumers often prefer “clear” products. Cosmetic agents which are preferred according to the invention are therefore characterized in that they are transparent or translucent.

For the purposes of the present invention, transparent or translucent is taken to mean a composition which has an NTU value of below 100. The NTU (Nephelometric Turbidity Unit) value is a unit used in water treatment for measuring turbidity in liquids. It is the unit which indicates the turbidity of a liquid measured with a calibrated nephelometer.

In a preferred embodiment of the invention, an agent according to the invention may furthermore also contain UV filters (I). UV filters to be used according to the invention are not subject to any general restrictions with regard to structure and physical properties. Rather, any UV filters usable in the field of cosmetics whose absorption maximum is in the UVA (315-400 nm), the UVB (280-315 um) or the UVC (<280 nm) range are suitable. UV filters with an absorption maximum in the UVB range, in particular in the range from approx. 280 to approx. 300 mu, are more preferred.

The UV filters are conventionally present in the agents according to the invention in quantities of 0.1-5 wt. % relative to the total agent. Quantities of 0.4-2.5 wt. % are preferred.

In a further preferred embodiment, the agents according to the invention may contain emulsifiers (F). Emulsifiers bring about the formation of water- or oil-resistant adsorption layers at the phase interface, which protect the dispersed droplets from coalescence and so stabilize the emulsion. Emulsifiers, like surfactants, are thus made up of a hydrophobic and a hydrophilic molecular moiety. Hydrophilic emulsifiers preferably form O/W emulsions while hydrophobic emulsifiers preferably form W/O emulsions. An emulsion is taken to mean a droplet distribution (dispersion) of one liquid in another liquid with the input of energy to create stabilizing phase interfaces by means of surfactants. The selection of these emulsifying surfactants or emulsifiers is here determined on the basis of the substances to be dispersed and the particular external phase and the fineness of the emulsion. Examples of emulsifiers which may be used according to the invention are:
addition products of 4 to 30 mol of ethylene oxide and/or 0 to 5 mol of propylene oxide onto linear fatty alcohols with 8 to 22 C atoms, onto fatty acids with 12 to 22 C atoms and onto alkylphenols with 8 to 15 C atoms in the alkyl group,
C₁₂-C₂₂ fatty acid mono- and diesters of addition products of 1 to 30 mol of ethylene oxide onto polyols with 3 to 6 carbon atoms, in particular onto glycerol, ethylene oxide and polyglycerol addition products onto methyl glucoside/fatty acid esters, fatty acid alkanolamides and fatty acid glucamides,
C₈-C₂₂ alkyl mono- and oligoglycosides and the ethoxylated analogs thereof, wherein degrees of oligomerization of 1.1 to 5, in particular of 1.2 to 2.0, and glucose as the sugar component are preferred,
mixtures of alkyl (oligo)glucosides and fatty alcohols, for example the commercially available product Montanov®68,
addition products of 5 to 60 mol of ethylene oxide onto castor oil and hardened castor oil,
partial esters of polyols having 3-6 carbon atoms with saturated fatty acids having 8 to 22 C atoms,
sterols. Sterols are taken to be a group of steroids which bear a hydroxyl group on C atom 3 of the steroid skeleton and may be isolated both from animal tissue (zoosterols) and from vegetable fats (phytosterols). Examples of zoosterols are cholesterol and lanosterol. Examples of suitable phytosterols are ergosterol, stigmasterol and sitosterol. Sterols are also isolated from fungi and yeasts, these being known as mycosterols.
phospholipids. These are primarily taken to mean glucose phospholipids which are for example obtained as lecithins, or phosphatidylcholines for example from egg yolk or plant seeds (for example soy beans).
fatty acid esters of sugars and sugar alcohols, such as sorbitol,
polyglycerols and polyglycerol derivatives such as for example polyglycerol poly-12-hydroxystearate (commercial product Dehymuls® PGPH),
linear and branched fatty acids having 8 to 30 C atoms and the Na, K, ammonium, Ca, Mg and Zn salts thereof.

The agents according to the invention preferably contain the emulsifiers in quantities of 0.1-25 wt. %, in particular of 0.5-15 wt. %, relative to the total agent.

The compositions according to the invention may preferably contain at least one nonionogenic emulsifier with an HLB value of 8 to 18. Nonionogenic emulsifiers with an HLB value of 10-15 may be more preferred according to the invention.

It has furthermore proven advantageous if, in addition to the polymer(s) from the group of cationic and/or amphoteric polymers, further polymers (G) are present in the agents according to the invention. In a preferred embodiment, the polymers are therefore added to the agents according to the invention, wherein both anionic and nonionic polymers have proven effective.

The anionic polymers (G2) are anionic polymers which comprise carboxylate and/or sulfonate groups. Examples of anionic monomers of which such polymers may consist are acrylic acid, methacrylic acid, crotonic acid, maleic anhydride and 2-acrylamido-2-methylpropanesulfonic acid. In this case, the acidic groups may be present wholly or in part as a sodium, potassium, ammonium, mono- or triethanolammonium salt. 2-Acrylamido-2-methylpropanesulfonic acid and acrylic acid are preferred monomers.

Anionic polymers which have proven very particularly effective are those which contain as sole or co-monomer 2-acrylamido-2-methylpropanesulfonic acid, wherein the sulfonic acid group may be present wholly or in part as a sodium, potassium, ammonium, mono- or triethanolammonium salt.

The homopolymer of 2-acrylamido-2-methylpropanesulfonic acid is more preferred, and is commercially obtainable for example under the name Rheothik® 11-80.

Within this embodiment it may be preferable to use copolymers of at least one anionic monomer and at least one nonionogenic monomer. With regard to anionic monomers, reference is made to the above-listed substances. Preferred nonionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and in particular polyacrylamide copolymers with monomers containing sulfonic acid groups. A particularly preferred anionic copolymer consists of 70 to 55 mol % acrylamide and 30 to 45 mol % 2-acrylamido-2-methylpropanesulfonic acid, wherein the sulfonic acid group is present wholly or in part as a sodium, potassium, ammonium, mono- or triethanolammonium salt. This copolymer may also be present in crosslinked form, wherein polyolefinically unsaturated compounds such as tetraallyloxyethane, allyl sucrose, allyl pentaerythritol and methylene bisacrylamide are preferably used as the crosslinking agents. Such a polymer is present in the commercial product Sepigel® 305 from SEPPIC. Use of this compound, which contains in addition to the polymer component a hydrocarbon mixture (C₁₃-C₁₄ isoparaffin) and a nonionogenic emulsifier (Laureth-7), has proven particularly advantageous for the purposes of the teaching according to the invention.

The sodium acryloyldimethyl taurate copolymers distributed under the name Simulgel® 600 as a compound with isohexadecane and polysorbate-80 have also proven particularly effective according to the invention.

Anionic homopolymers which are likewise preferred are uncrosslinked and crosslinked polyacrylic acids. In this case, allyl ethers of pentaerythritol, of sucrose and of propylene may be preferred crosslinking agents. Such compounds are commercially obtainable for example under the trademark Carbopol®.

Copolymers of maleic anhydride and methyl vinyl ether, in particular those comprising crosslinks, are also color-preserving polymers. A maleic acid-methyl vinyl ether copolymer crosslinked with 1,9-decadiene is commercially obtainable under the name Stabileze® QM. In a further embodiment, the agents according to the invention may contain nonionogenic polymers (G4).

Suitable nonionogenic polymers are for example:
vinylpyrrolidone/vinyl ester copolymers, as are distributed for example under the tradename Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methylhydroxypropylcellulose, as are distributed for example under the tradenames Culminal® and Benecel® (AQUALON) and Natrosol® grades (Hercules).
starch and the derivatives thereof, in particular starch ethers, for example Structure® XL (National Starch), a multifunctional, salt-tolerant starch;
shellac
polyvinylpyrrolidones, as are distributed for example under the tradename Luviskol® (BASF).
siloxanes. These siloxanes may be either water-soluble or water-insoluble. Both volatile and non-volatile siloxanes are suitable, wherein non-volatile siloxanes are taken to be those compounds whose boiling point at standard pressure is above 200° C. Preferred siloxanes are polydialkylsiloxanes, such as for example polydimethylsiloxane, polyalkylarylsiloxanes, such as for example polyphenylmethylsiloxane, ethoxylated polydialkylsiloxanes and polydialkylsiloxanes, which contain amine and/or hydroxy groups.
glycosidically substituted silicones.

It is also possible according to the invention for the preparations to contain a plurality of, in particular two different, identically charged polymers and/or in each case one ionic and one amphoteric and/or nonionic polymer.

The further polymers (G) are present in the agents according to the invention preferably in quantities of from 0.05 to 10 wt. %, relative to the total agent. Quantities of 0.1 to 5, in particular of 0.1 to 3 wt. %, are more preferred.

The present invention also provides a method for treating keratinic fibers, in which a hair treatment agent according to the invention is applied onto the keratinic fibers and, after a period of exposure of a few seconds up to 45 minutes, rinsed back out.

The above statements regarding the agents according to the invention apply mutatis mutandis with regard to preferred embodiments of the methods according to the invention.

The present invention also provides the use of hair treatment agents according to the invention
for conditioning keratin substances and/or
in order to improve volume, softness, gloss and/or combability and to facilitate styling of keratin substances and/or
in order to improve the persistence of the conditioning action on hair washing and/or
for improving wet and dry combability and/or
for improving gloss and/or
for improving the moisture balance of keratinic fibers and/or
for protecting keratinic fibers from oxidative damage and/or
for preventing keratinic fibers from becoming greasy again and/or
for increasing the washing resistance of dyed keratinic fibers.

The above statements regarding the agents according to the invention also apply mutatis mutandis with regard to further preferred embodiments of the uses according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition containing in a cosmetically acceptable medium
(a) at least one conditioning agent selected from synthetic oils, mineral oils, plant oils, fluorinated or perfluorinated oils, natural or synthetic waxes, compounds of the ceramide type, carboxylic acid esters, silicones differing from the silicones of formulae (I), anionic polymers, nonionic polymers, cationic polymers, amphoteric polymers, cationic proteins, cationic protein hydrolysates, cationic interfacially active substances and mixtures of these various compounds, and
(b) at least one hydroxy-terminated organopolysiloxane of general formula (I), 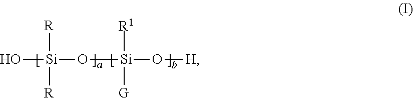
wherein
R means a monovalent unsubstituted or halo-substituted hydrocarbon residue with 1 to 20 carbon atoms,
R1 means a monovalent unsubstituted or halo-substituted hydrocarbon residue with 1 to 20 carbon atoms, —OR4 or —OH,
R4 means an alkyl residue with 1 to 6 carbon atoms,
G means a group of general formula (II) 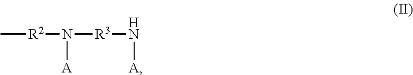
wherein
R2, R3 mutually independently mean a divalent hydrocarbon residue with 1 to 6 carbon atoms, wherein non-adjacent —CH2 units may be replaced by units which are selected from —C(═O)—, —O—, and —S—,
A means R5-C(═O)—,
R5 means an alkyl residue with 1 to 20 carbon atoms,
a means integral values of 100 to 1500 and
b means integral values of at least 1.

2. The cosmetic composition according to claim 1, wherein the residue R is selected from the group consisting of: methyl, ethyl, n-propyl, isopropyl, n-butyl, isobutyl, sec-butyl, tert-butyl, n-pentyl, and n-hexyl residues.

3. The cosmetic composition according to claim 1, wherein the residues R2, R3 are selected from ethylene, n-propylene, iso-butylene or n-butylene residues.

4. The cosmetic composition according to claim 1, wherein the R5 residue is selected from the group consisting of methyl, ethyl, n-propyl, isopropyl, n-butyl, isobutyl, sec-butyl, tert-butyl, n-pentyl, and n-hexyl residues.

5. The cosmetic composition according to claim 1, wherein the R5 residue is a methyl or ethyl residue.

6. The cosmetic composition according to claim 1, wherein the hydroxy-terminated organopolysiloxane(s) of general formula (I) comprises 0.00001 to 10 wt. % of the composition.

7. The cosmetic composition of claim 1, wherein the hydroxyl terminated organopolysiloxane of general formula (I) comprises 0.0001 to 7.5 wt. % of the composition.

8. The cosmetic composition according to claim 1, wherein the composition comprises 0.00001 to 5 wt. % branched ethoxylated tridecanol or α-iso-tridecyl-ω-hydroxy polyglycol ether, or mixtures thereof.

9. The cosmetic composition according to claim 1, wherein the weight-average molar mass of the hydroxy-terminated organopolysiloxane of general formula (I) is 2,000 to 1,000,000 gmol-1.

10. The cosmetic composition according to claim 1, wherein the hydroxy-terminated organopolysiloxane of general formula (I) is present in the composition as an oil-in-water emulsion in which the number-average size of the silicone particles in the emulsion is in the range from 3 to 500 nm.

11. The cosmetic composition according to claim 1, wherein the at least one conditioning agent includes a plant oil selected from the group consisting of: sunflower oil, corn oil, soy oil, avocado oil, jojoba oil, pumpkin seed oil, grapeseed oil, sesame oil, hazelnut oil, fish oils, glycerol tricaprocaprylate, plant oils of formula R9COOR10, in which R9 means the residue of a higher fatty acid with 7 to 29 carbon atoms and R10 means a linear or branched hydrocarbon chain with 3 to 30 carbon atoms, and natural or synthetic essential oils.

12. The cosmetic composition according to claim 1, wherein the at least one conditioning agent includes a wax selected from the group consisting of: carnauba wax, candelilla wax, alfa wax, paraffin wax, ozokerite, plant waxes, animal waxes, polyethylene waxes, and polyolefin waxes.

13. The cosmetic composition according to claim 1, wherein conditioning agent includes a carboxylic acid esters selected from the group consisting of: ethyl palmitate, isopropyl palmitate, 2-ethylhexyl palmitate, 2-octyldecyl palmitate, alkyl myristates, hexyl stearate, butyl stearate, isobutyl stearate; dioctyl malate, hexyl laurate, 2-hexyldecyl laurate, isononyl isononanoate and cetyl octanoate.

14. The cosmetic composition according to claim 1, characterized in that the silicones used individually or in combination are selected from the group consisting of:
polydimethylsiloxane,
polydimethylsiloxane/methylvinylsiloxanes,
polydimethylsiloxane/diphenylsiloxane,
polydimethylsiloxane/phenylmethylsiloxane,
polydimethylsiloxane/diphenylsiloxane/methylvinylsiloxane and the following mixtures:
mixtures composed of a chain end-hydroxylated polydimethylsiloxane and a cyclic polydimethylsiloxane,
mixtures composed of a polydimethylsiloxane gum and a cyclic silicone, and
mixtures of polydimethylsiloxanes of various viscosities.

15. The cosmetic composition according to claim 1, wherein the conditioning agent(s) comprises 0.001 to 20 wt. % relative to the total weight of the composition.

16. A method for treating keratinic fibers, characterized in that the hair treatment agent according to claim 1 is applied onto the keratinic fibers and, after a period of exposure of a few seconds up to 45 minutes, rinsed back out.
